(19) United States
(12) Patent Application Publication
(10) Pub. No.: US 2026/0266803 A1
(43) Pub. Date: Sep. 10, 2026

(54) Induced Pluripotent Stem Cell Model of Fibrous Dysplasia/Mccune-Albright Syndrome and Methods of Use

(71) Applicant: The Regents of the University of California, Oakland, CA (US)

(72) Inventors: Edward Hsiao, San Francisco, CA (US); Kelly Wentworth, San Francisco, CA (US)

(21) Appl. No.: 19/166,409

(22) PCT Filed: Mar. 28, 2024
(86) PCT No.: PCT/US2024/021912

§ 371 Date: Sep. 17, 2025

Related U.S. Application Data
(60) Provisional application No. 63/493,645, filed on Mar. 31, 2023.

(51) Int. Cl. G01N 33/50; C07K 14/47; C12N 5/074; C12N 9/22; C12N 15/11; C12Q 1/527

(52) U.S. Cl. CPC G01N 33/5023; C07K 14/4702; C12N 5/0696; C12N 9/222; C12N 15/111; C12Q 1/527; G01N 33/5044; C12N 2310/20; C12N 2510/00; G01N 2333/988; G01N 2500/10

(57) ABSTRACT

Compositions and methods are provided for screening for an agent that inhibits activity of a hyperactive stimulatory guanine nucleotide binding protein alpha subunit (Gsa) for use in treating disorders associated with GNAS hyperactivation. In particular, induced pluripotent stem cells (iPSCs) are provided for therapeutic screening that comprise a genetically modified GNAS gene comprising an activating mutation, wherein the genetically modified GNAS gene comprises a coding sequence encoding a hyperactive stimulatory guanine nucleotide binding protein alpha subunit (Gsa). The GNAS activating mutation may be linked to fibrous dysplasia, McCune-Albright syndrome, cancer, benign tumors, or other GNAS-associated disorders.

### CROSS-REFERENCE TO RELATED APPLICATIONS

This application is a 371 of PCT/US2024/021912 filed Mar. 28, 2024, which claims the benefit of priority to U.S. Provisional Application Ser. No. 63/493,645, filed Mar. 31, 2023, the contents which are hereby incorporated by reference in their entirety.

### STATEMENT REGARDING FEDERALLY SPONSORED RESEARCH OR DEVELOPMENT

This invention was made with government support under K08 DE028946 awarded by The National Institutes of Health. The government has certain rights in the invention.

### INCORPORATION BY REFERENCE OF A SEQUENCE LISTING

A Sequence Listing is provided herewith as a Sequence Listing XML file, “UCSF-690WO_SEQ_LIST”, created on Mar. 27, 2024, and having a size of 17,978 bytes. The contents of the Sequence Listing XML file are incorporated by reference herein in their entirety.

### BACKGROUND OF THE INVENTION

GPCRs mediate a wide variety of biological processes (Fredriksson et al. (2003) Mol Pharmacol. 63(6), 1256-1272; Karchin et al. (2002) Bioinformatics 18(1), 147-159). The diversity of GPCRs and their responses to small molecules have made them major targets for over 40% of modern pharmaceuticals (5). While GPCRs are strongly implicated in development (7-9), their precise roles in tissue differentiation are still being defined.

GPCRs signal through a select number of canonical pathways (Gether (2000) Endocr. Rev. 21(1), 90-113): the Gₛ and Gᵢ pathways increase or decrease intracellular cyclic AMP (cAMP) levels, respectively, by acting on adenylate cyclase, whereas the G_{q} pathway increases intracellular calcium by activating phospholipase C. Multiple GPCRs are expressed in bone (Juppner et al. (1991). Science 254(5034), 1024-1026; Kasperk et al. (1997) Calcif. Tissue Int 60(4), 368-374; Suzawa et al. (2000) Endocrinology 141(4), 1554-1559; Moore et al. (1993) Bone Miner. 23(3), 301-315; Togari et al. (1997) Neurosci. Lett. 233(2-3), 125-128; Abe et al. (2003) Cell 115(2), 151-162). The parathyroid hormone (PTH) receptor (PTHR1), which signals primarily via Gₛ, is the best-studied GPCR in bone formation and function. Polymorphisms in PTHR1 that affect the signaling properties of the receptor are linked to variations in bone mineral density (Vilarino-Guell et al. (2007) Calcif Tissue Int 81(4), 270-278). Mice expressing a constitutively active Gₛ-coupled PTHR1 in osteoblasts show increased trabecular bone volume and decreased cortical bone thickness at 12 weeks of age, with grossly normal femur shape and size (Calvi et al. (2001) J. Clin. Invest. 107(3), 277-286). Models using PTH peptide fragments that selectively activate PTHR1-linked Gₛ signaling (Rixon et al. (1994) J. Bone Miner. Res. 9(8), 1179-1189; Armamento-Villareal et al. (1997) J. Bone Miner. Res. 12(3), 384-392; Hilliker et al. (1996) Bone 19(5), 469-477; Whitfield et al. (1996) Calcif. Tissue Int. 58(2), 81-87) suggest the bone anabolic effects of PTHR1 are regulated via Gₛ (Armamento-Villareal (1997), supra; Whitfield (1996), supra). PTH also stimulates osteoblast differentiation in immature osteoblasts but inhibits further differentiation in more mature cells (Isogai et al. (1996) Journal of Bone and Mineral Research: the Official Journal of the American Society for Bone and Mineral Research 11(10), 1384-1393). In humans, recombinant PTH/PTHrP analogs (e.g., teriparatide, abaloparatide) are important therapies that harness the anabolic role of Gₛ signaling for promoting bone repair and increasing bone mass to treat osteoporosis (Dobnig and Turner (1997) Endocrinology 138(11), 4607-4612; Neer et al. (2001) N. Engl. J. Med. 344(19), 1434-1441) and speed fracture repair. Gₛ-GPCR signaling dramatically affects the bone niche, including hematopoietic stem cell function (Schepers et al. (2012) Blood 120(17), 3425-3435; Wu, et al. (2009) J Bone Miner Res 24(5), 759-764; Calvi et al. (2003) Nature 425(6960), 841-846), fracture repair (Wang et al. (2015) J Bone Miner Res 30(10), 1896-1904; Alkhiary et al. (2005) J Bone Joint Surg Am 87(4), 731-741), and osteogenic cell fate (Jilka (2007) Bone 40(6), 1434-1446; van der Horst et al. (2005) J. Bone Miner. Res. 20(12), 2233-2244), through direct GPCR signaling and pathways, such as Wnt (Regard et al. (2012) Cold Spring Harb. Perspect. Biol. 4(12); Khan et al. (2018) Proc. Natl. Acad. Sci. U.S.A. 115(3), E418-E427), Hedgehog (Pan et al. (2013) Frontiers in Physiology 4, 61), and Yap/Taz (Yu et al. (2012) Cell 150(4), 780-791). In addition, many receptors, including PTHR1, activate all three major GPCR pathways (Gₛ, Gᵢ, and G_{q}) (Abou-Samra et al. (1992) Proc Natl. Acad. Sci. U.S.A. 89(7), 2732-2736; Bringhurst et al. (1993) Endocrinology 132(5), 2090-2098; Gensure et al. (2005) Biochem. Biophys. Res. Commun. 328(3), 666-678), so directed targeting of the Gₛ pathway is attractive to avoid cross-activation. Notably, increased Wnt signaling is an essential mediator of the skeletal dysplasia produced by activated Gₛ, but how Gₛ-GPCR signaling activates the Wnt pathway and the respective cell fate changes remain poorly understood (Regard et al. (2011) Proc. Natl. Acad. Sci. U.S.A. 108(50), 20101-20106; Remoli et al. (2015) Journal of Bone and Mineral Research: the Official Journal of the American Society for Bone and Mineral Research 30(6), 1030-1043). Thus, knowing how Gₛ-signaling affects skeletal cells will reveal key physiology relevant to tissue repair and regeneration.

### SUMMARY OF THE INVENTION

Compositions and methods are provided for screening for an agent that inhibits activity of a hyperactive stimulatory guanine nucleotide binding protein alpha subunit (Gsa) for use in treating disorders associated with GNAS hyperactivation. In particular, induced pluripotent stem cells (iPSCs) are provided for therapeutic screening that comprise a genetically modified GNAS gene comprising an activating mutation, wherein the genetically modified GNAS gene comprises a coding sequence encoding a hyperactive stimulatory guanine nucleotide binding protein alpha subunit (Gsa). The GNAS activating mutation may be linked to fibrous dysplasia, McCune-Albright syndrome, cancer, benign tumors, or other GNAS-associated disorders.

In one aspect, a method of screening for an agent that inhibits activity of a hyperactive Gsa for use in treating a guanine nucleotide-binding protein alpha, stimulatory (GNAS)-associated disorder is provided, the method comprising: providing an iPSC comprising a genetically modified GNAS gene comprising an activating mutation, wherein the genetically modified GNAS gene comprises a coding sequence encoding a hyperactive Gsa; contacting the iPSC with a candidate agent; and measuring inhibition of the hyperactive Gsa by the candidate agent.

In certain embodiments, the method further comprises contacting an iPSC comprising an unmodified GNAS gene encoding a wild-type Gsa with the candidate agent; measuring inhibition of activity of the wild-type Gsa by the candidate agent; comparing levels of inhibition of the wild-type Gsa and the hyperactive Gsa, wherein increased inhibition of the hyperactive Gsa compared to the wild-type Gsa indicate that the candidate agent selectively inhibits the hyperactive Gsa.

In certain embodiments, the GNAS-associated disorder is fibrous dysplasia, McCune-Albright syndrome, cancer, or a benign tumor.

In certain embodiments, the activating mutation is linked to fibrous dysplasia or McCune-Albright syndrome.

In certain embodiments, the hyperactive Gsa comprises a substitution of histidine or cysteine at amino acid position 201, wherein numbering of amino acid positions is relative to the reference amino acid sequence of SEQ ID NO:1.

In certain embodiments, the genetically modified GNAS gene comprises a substitution of thymine at nucleotide position 601 or adenine at nucleotide position 602, wherein numbering of nucleotide positions is relative to the reference nucleotide sequence of SEQ ID NO:2. In some embodiments, the genetically modified GNAS gene further comprises a substitution of thymine at nucleotide position 600, wherein numbering of nucleotide positions is relative to the reference nucleotide sequence of SEQ ID NO:2.

In certain embodiments, the genetically modified GNAS gene comprises a nucleotide sequence selected from the group consisting of SEQ ID NOS:3-8, or a nucleotide sequence having at least about 80-100% sequence identity to a nucleotide sequence selected from the group consisting of SEQ ID NOS:3-8, including any percent identity within this range, such as 81%, 82%, 83%, 84%, 85%, 86%, 87%, 88%, 89%, 90%, 91%, 92%, 93%, 94%, 95%, 96%, 97%, 98%, or 99% sequence identity thereto, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

In certain embodiments, a CRISPR system is used to genetically modify the genome of the iPSC to introduce the activating mutation into the GNAS gene, wherein a hyperactive Gsa is expressed in the iPSC. In some embodiments, the CRISPR system comprises a guide RNA (gRNA) capable of hybridizing to a target site in the GNAS gene. In some embodiments, the CRISPR system comprises a Cas9 nuclease.

In certain embodiments, measuring inhibition of the Gsa comprises measuring inhibition of activation of adenylate cyclase activity by the hyperactive Gsa. In some embodiments, measuring inhibition of activation of adenylate cyclase activity comprises measuring levels of cyclic adenosine monophosphate (cAMP) in the presence and absence of a candidate agent, wherein reduced levels of cAMP in the presence of the candidate agent compared to in the absence of the candidate agent indicate that the candidate agent inhibits activation of adenylate cyclase activity by the hyperactive Gsa.

In certain embodiments, measuring inhibition of the Gsa comprises measuring inhibition of GTPase activity of the Gsa.

In certain embodiments, the method further comprises contacting the iPSC with a β-adrenergic receptor agonist (e.g., isoproterenol); and measuring inhibition of the hyperactive Gsa by the candidate agent in presence of the β-adrenergic receptor agonist.

In certain embodiments, the method further comprises contacting the iPSC with a phosphodiesterase inhibitor; and measuring inhibition of the hyperactive Gsa by the candidate agent in presence of the phosphodiesterase inhibitor.

In certain embodiments, the method further comprises measuring binding of the candidate agent to the hyperactive Gsa.

In certain embodiments, the method further comprises measuring binding of the candidate agent to a G protein-coupled receptor (GPCR) that activates the hyperactive Gsa.

In certain embodiments, the candidate agent does not fully inhibit the wild-type Gsa.

In certain embodiments, the agent is a small molecule, a drug, a peptide, a protein, an aptamer, a peptoid, an antibody, or an antibody mimetic.

In another aspect, a composition comprising: an iPSC comprising a genetically modified GNAS gene comprising an activating mutation is provided, wherein the genetically modified GNAS gene comprises a coding sequence encoding a hyperactive Gsa.

In certain embodiments, the activating mutation is linked to fibrous dysplasia, McCune-Albright syndrome, or cancer.

In certain embodiments, the iPSC comprising a genetically modified GNAS gene encoding a hyperactive Gsa comprising a substitution of histidine or cysteine at amino acid position 201, wherein numbering of amino acid positions is relative to the reference amino acid sequence of SEQ ID NO:1.

In certain embodiments, the iPSC comprises a genetically modified GNAS gene comprising a substitution of thymine at nucleotide position 601 or adenine at nucleotide position 602, wherein numbering of nucleotide positions is relative to the reference nucleotide sequence of SEQ ID NO:2. In some embodiments, the genetically modified GNAS gene further comprises a substitution of thymine at nucleotide position 600, wherein numbering of nucleotide positions is relative to the reference nucleotide sequence of SEQ ID NO:2.

In certain embodiments, the iPSC comprising a genetically modified GNAS gene comprising a nucleotide sequence selected from the group consisting of SEQ ID NOS:3-8, or a nucleotide sequence having at least about 80-100% sequence identity to a nucleotide sequence selected from the group consisting of SEQ ID NOS:3-8, including any percent identity within this range, such as 81%, 82%, 83%, 84%, 85%, 86%, 87%, 88%, 89%, 90%, 91%, 92%, 93%, 94%, 95%, 96%, 97%, 98%, or 99% sequence identity thereto, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

In certain embodiments, the composition further comprises a β-adrenergic receptor agonist (e.g., isoproterenol).

In certain embodiments, the composition further comprises a phosphodiesterase inhibitor.

### BRIEF DESCRIPTION OF THE DRAWINGS

FIGS. 1A-1B. Fibrous dysplasia affecting the skull (axial CT). (FIG. 1A) A normal CT scan of the craniofacial bones, from a skeletally normal 61-year-old male. (FIG. 1B) A 29-year-old female with craniofacial FD, skull asymmetry [ground glass lesions, (*) including in left orbit (**), and displaced right eye globe because of extensive overlying FD (***)].

FIGS. 2A-2E. Rs1-mediated Gₛ activation in osteoblastic cells causes fibrous dysplasia. (FIG. 2A) 9-week old normal mouse. Mid-femur microCT cross sections are shown below. (FIG. 2B) 9-week-old Coll(2.3)⁺/Rs1⁺ mouse with increased trabecular bone resembling FD (3; 4). (FIG. 2C) Patient with severe FD showing similar femur trabecularization. Clinical image from M. Collins (6). (FIG. 2D) Calvaria from 1- and 9-week-old Coll(2.3)⁺/Rs1⁺ mice, showing increased calvarial bone formation (10). (FIG. 2E) Suppressing Gₛ-GPCR activity in osteoblasts can reverse the FD bone lesions (12). s, bone marrow space; c, cortical bone, t, trabecular bone. Scale bars: white=1 mm.

FIGS. 3A-3C. GNAS^{R201H} iPSCs show increased cAMP production. (FIG. 3A) GNAS^{R201H} iPSCs show increased cAMP production at baseline (red arrow) and with forskolin stimulation through 250 μM, compared to isogenic control iPSCs. Representative of n=3. (FIG. 3B) Representative western blot of pluripotent and differentiated (DDX) iPSCs showing increased pCREB and pATF1, consistent with activation of the cAMP pathway. (FIG. 3C) GNAS^{R201H} iPSCs treated with low dose (10 μM) isoproterenol showed increased differences in cAMP from baseline. The negative cAMP values in WT iPSCs are within the measurement error of the basal cAMP assay, demonstrating the low sensitivity of the assay. N=6 in two different iPSC lines.

FIGS. 4A-4B. Promising candidates from our initial screening. Compounds were screened using our control or GNAS^{R201H} iPSC lines, in the presence of a phosphodiesterase inhibitor and without forskolin. (FIG. 4A) Overlap of WT (white backbone) and R201H (teal) Gₛα structures of the active site show potential differences that can be exploited for selectivity. (FIG. 4B) Example candidate compounds tested in our human iPSC model system. Error bars are not shown for clarity, but significance from triplicates is indicated relative to vehicle-only controls (*, p<0.05; others not significant). red=GNAS^{R201H}; blue=GNAS^{WT} cells.

FIG. 5. An engineered human iPSC model of McCune-Albright syndrome (MAS). A CRISPR/Cas9 system was used to genetically modify the genome of iPSCs to introduce the activating mutation, c.602G>A (p.R201H), into an endogenous GNAS gene.

FIG. 6. GNAS construct (SEQ ID NO:7 and SEQ ID NO:8) showing sites of GNAS mutations, including p.R201C (c.601C>T), p.R201H (c.602G>A), and a silent mutation c.600 C>T used to facilitate detection of the transcript, and restriction sites.

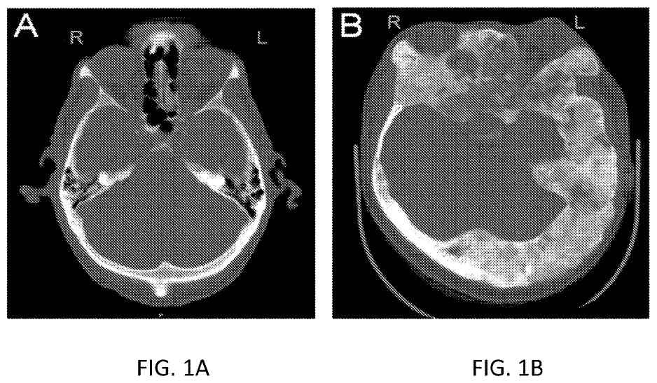

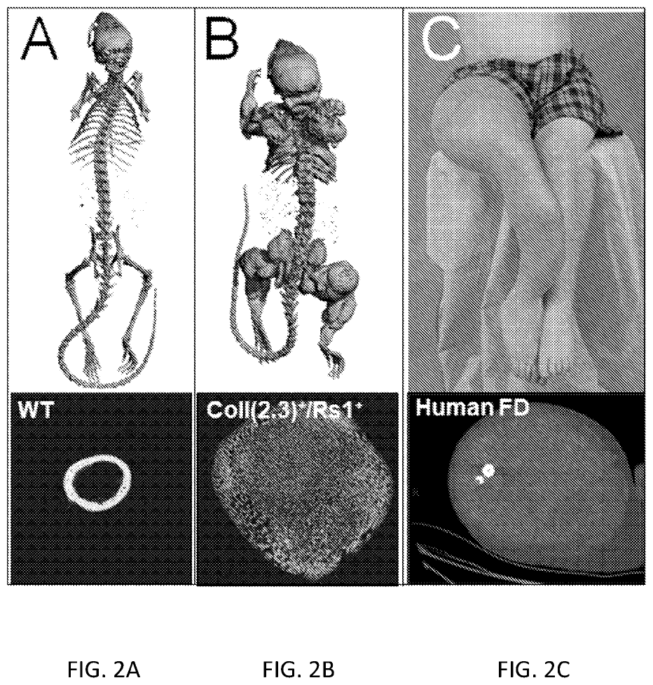

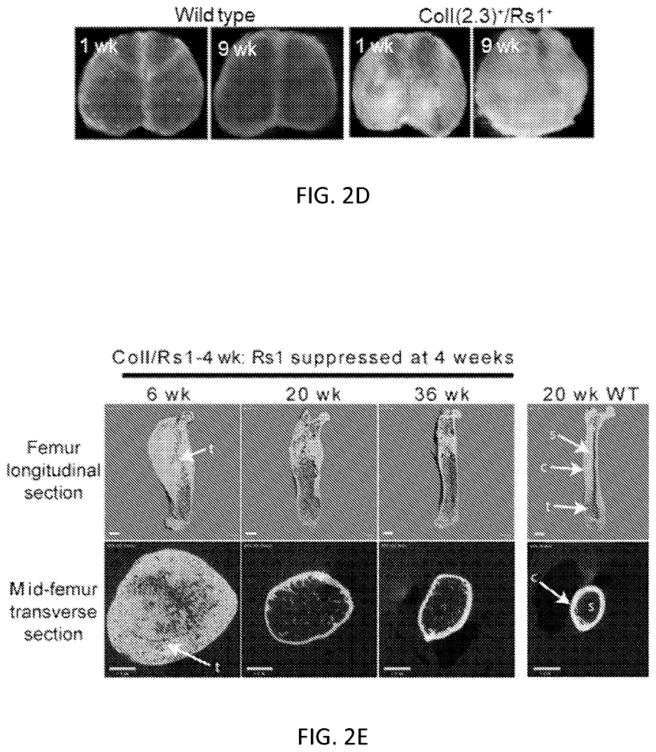

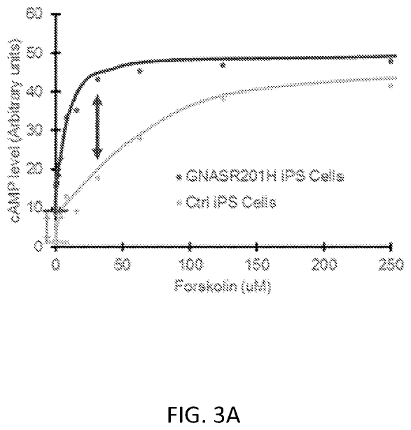

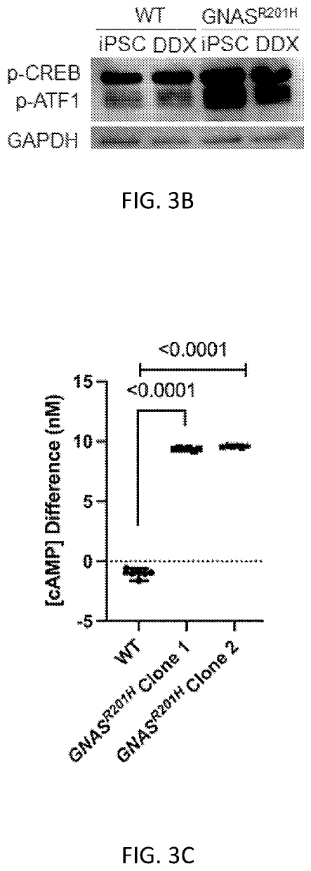

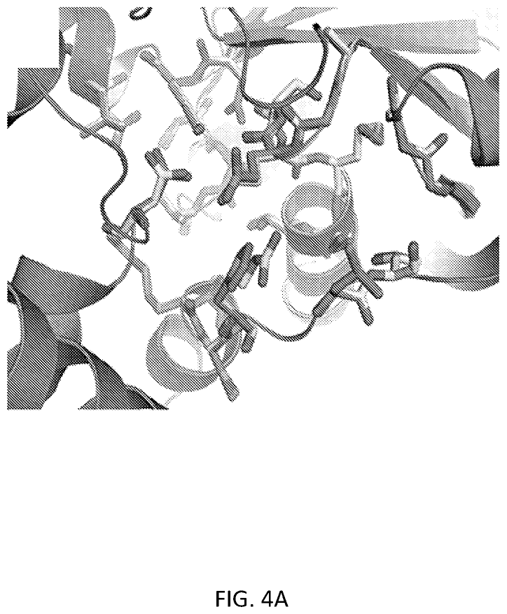

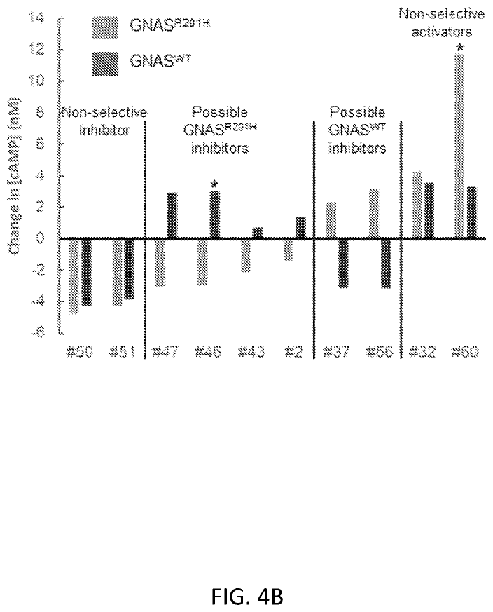

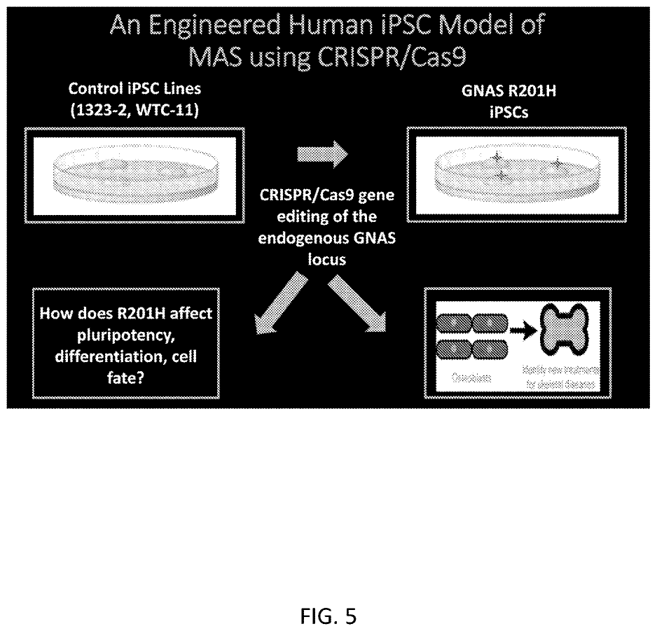

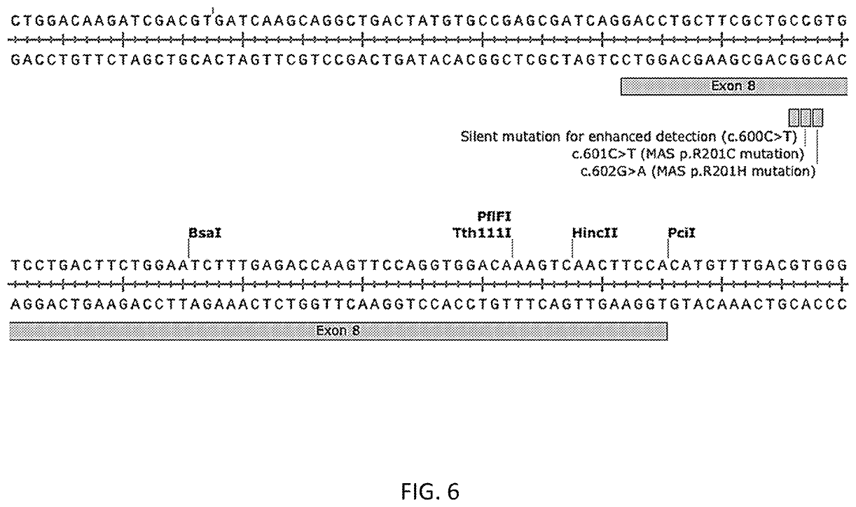

### DETAILED DESCRIPTION OF THE INVENTION

Compositions and methods are provided for screening for an agent that inhibits activity of a hyperactive stimulatory guanine nucleotide binding protein alpha subunit (Gsa) for use in treating disorders associated with GNAS hyperactivation. In particular, iPSCs are provided for therapeutic screening that comprise a genetically modified GNAS gene comprising an activating mutation, wherein the genetically modified GNAS gene comprises a coding sequence encoding a hyperactive stimulatory guanine nucleotide binding protein alpha subunit (Gsa). The GNAS activating mutation may be linked to fibrous dysplasia, McCune-Albright syndrome, cancer, benign tumors, or other GNAS-associated disorders.

Before the present compositions and methods are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the induced pluripotent stem cell” includes reference to one or more induced pluripotent stem cells and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The term “hyperactive or “activated”, as used herein with reference to a Gsα protein, refers to a Gsα protein having a modification to its sequence resulting in increased Gsα biological activity. One or more Gsα biological activities may be enhanced by a mutation, including GTP binding, GTP hydrolysis, and/or activation of adenylate cyclase, which increases production of intracellular cAMP, and/or association with downstream effectors that mediate Gsα effects on various biological events, and/or other biological activities identifiable by a skilled person.

A hyperactive Gsα protein may have one or more mutations that increase one or more biological activities of a Gsα protein. In some embodiments, the hyperactive Gsα protein has a mutation (e.g., a point mutation) at an amino acid residue corresponding to amino acid residue number 201, wherein numbering of amino acid positions is relative to the reference amino acid sequence of SEQ ID NO 1. In some embodiments, a hyperactive Gsα comprises a substitution of histidine or cysteine at amino acid position 201, wherein numbering of amino acid positions is relative to the reference amino acid sequence of SEQ ID NO:1.

The term “activated” or “activating” as used herein with reference to a mutation in a GNAS gene indicates a GNAS gene encoding a hyperactive Gsα protein in the sense of the disclosure. In some embodiments, the GNAS gene comprises an activating mutation (e.g., c.602G>A (p.R201H) or c.601C>T (p.R201C)) resulting in expression of a hyperactive or constitutively active Gsα protein.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

As used herein, the terms “increase,” “increasing,” “improve” and “improving” (and grammatical variations thereof) describes, unless the context indicates otherwise, a detectable elevation of a reference value. An increase can comprise an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

“Substantially purified” generally refers to isolation of a substance (e.g., compound, drug, polynucleotide, protein, polypeptide, peptide, antibody, antibody mimetic, aptamer, peptoid, inhibitor) such that the substance comprises the majority percent of the sample in which it resides. Typically in a sample, a substantially purified component comprises 50%, preferably 80%-85%, or more preferably 90-95% of the sample.

The term “GNAS inhibitor” or “Gsα inhibitor” as used herein refers to any molecule (e.g., small molecule inhibitor, drug, protein, polypeptide, peptide, fusion protein, peptide nucleic acid, peptoid, antibody, antibody mimetic, or aptamer) that inhibits biological activity of a hyperactive stimulatory guanine nucleotide binding protein alpha subunit (Gsa) comprising an activating mutation (e.g., R201H, R201C, and Q227L). The inhibitor may inhibit GTPase activity of the hyperactive Gsα and/or inhibit activation of adenylate cyclase activity by the hyperactive Gsα and/or reduce intracellular levels of cyclic adenosine monophosphate (cAMP). Inhibition may be complete or partial (i.e., all activity, some activity, or most activity is blocked by an inhibitor). For example, an inhibitor may reduce the activity of the hyperactive Gsα by 10%, 20%, 30%, 40%, 50%, 60%, 70%, 80%, 90%, 100%, or any amount in between as compared to native or control levels. Preferably, the inhibitor selectively inhibits a hyperactive Gsα comprising an activating mutation and does not inhibit or inhibits to a lesser extent a wild-type Gsα.

An “effective amount” of an inhibitor of a hyperactive Gsα (e.g., small molecule inhibitor, drug, protein, polypeptide, peptide, fusion protein, peptide nucleic acid, peptoid, antibody, antibody mimetic, or aptamer) is an amount sufficient to inhibit the biological activity of a hyperactive Gsα comprising an activating mutation, for example, by inhibiting GTPase activity of the hyperactive Gsα and/or inhibiting activation of adenylate cyclase activity by the hyperactive Gsα and/or reduce intracellular levels of cAMP. An effective amount can be administered in one or more administrations, applications, or dosages.

The terms “specific binding,” “specifically binds,” “selectively binds,” and the like, refer to non-covalent or covalent preferential binding to a molecule relative to other molecules or moieties in a solution or reaction. In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less). “Affinity” refers to the strength of binding, increased binding affinity being correlated with a lower K_{D}. In an embodiment, affinity is determined by surface plasmon resonance (SPR), e.g., as used by Biacore systems. The affinity of one molecule for another molecule is determined by measuring the binding kinetics of the interaction, e.g., at 25° C.

By “test agent,” “candidate agent,” and grammatical equivalents herein, which terms are used interchangeably herein, is meant any molecule including macromolecules (e.g., proteins, antibodies, polynucleotides), small molecules (e.g., 5-1000 Da, 100-750 Da, 200-500 Da, or less than 500 Da in size), organic or inorganic molecules, drugs, etc. that are to be tested for activity (e.g., binding and inhibiting a hyperactive Gsα) in a subject assay.

“GNAS-associated disorders” include any disorder linked to a mutated GNAS gene comprising an activating mutation (e.g., c.602G>A (p.R201H) or c.601C>T (p.R201C)) and expressing a hyperactive or constitutively active Gsα. GNAS-associated disorders include, but are not limited to, fibrous dysplasia, McCune-Albright syndrome, cancers such as, but not limited to, breast cancer (e.g., breast invasive ductal carcinoma), pancreatic cancer (e.g., pancreatic adenocarcinoma), adrenal cancer, lung cancer (e.g., lung adenocarcinoma, squamous cell lung cancer, small cell lung cancer, and non-small cell lung cancer), colon cancer (e.g., colon adenocarcinoma), rectal cancer (e.g., rectal adenocarcinoma), prostate cancer (e.g., adenocarcinoma), bladder cancer, endometrial cancer, esophageal cancer (e.g., esophageal adenocarcinoma), gastric cancer (e.g., gastric adenocarcinoma), hepatocellular carcinoma, glioblastoma, appendix mucinous adenocarcinoma, cutaneous melanoma, leukemia, and Pseudomyxoma peritonei; and benign tumors such as, but not limited to, intraductal papillary mucinous neoplasms, myxomas, and adenomas.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with a GNAS-associated disorder) as well as those in which prevention is desired (e.g., those with increased susceptibility to a GNAS-associated disorder, those with a genetic predisposition to developing a GNAS-associated disorder, those suspected of having a GNAS-associated disorder, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, or hepatocytes.

As used herein, “reprogramming factors” refers to one or more, i.e., a cocktail, of biologically active factors that act on a cell to alter transcription, thereby reprogramming a cell to multipotency or to pluripotency. Reprogramming factors may be provided individually or as a single composition, that is, as a premixed composition, of reprogramming factors to the cells, e.g., somatic cells from an individual with a family history or genetic make-up of interest, such as a patient who has a neurological disorder or a neurodegenerative disease. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

The somatic cells may include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, or hepatocytes, etc., which are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector.

The somatic cells or the IPSCs derived therefrom may be genetically modified for a variety of purposes, e.g., to introduce an activating mutation (e.g., c.602G>A (p.R201H) or c.601C>T (p.R201C)) into a GNAS gene. Vectors may be introduced that express an exogenous gene, reprogramming factors, CRISPR systems, antisense nucleic acids, or ribozymes. Various techniques known in the art may be used to introduce nucleic acids into the target cells, e.g., electroporation, calcium precipitated DNA, fusion, transfection, lipofection, infection and the like. The particular manner in which the DNA is introduced is not critical to the practice of the invention.

By “container” is meant a glass, plastic, or metal vessel that can provide an aseptic environment for culturing cells.

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

The terms “subject”, “individual” or “patient” are used interchangeably herein and refer to a vertebrate, preferably a mammal. By “vertebrate” is meant any member of the subphylum Chordata, including, without limitation, humans and other primates, including nonhuman primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

A “CRISPR system” refers collectively to transcripts and other elements involved in the expression of or directing the activity of CRISPR-associated (“Cas”) genes. In some embodiments, one or more elements of a CRISPR system is derived from a type I, type II, or type Ill CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks).

A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

By “selectively binds” with reference to a guide RNA is meant that the guide RNA binds preferentially to a target sequence of interest or binds with greater affinity to the target sequence than to other genomic sequences. For example, a gRNA will bind to a substantially complementary sequence and not to unrelated sequences. A gRNA that selectively binds to a particular target DNA sequence will selectively direct binding of Cas9 to a substantially complementary sequence at the target site and not to unrelated sequences.

The term “donor polynucleotide” refers to a polynucleotide that provides a sequence of an intended edit to be integrated into the genome at a target locus by homology directed repair (HDR).

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele).

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. The nucleotide sequence comprising the intended edit is integrated into the genomic DNA by HDR or recombineering at the genomic target locus recognized (i.e., sufficiently complementary for hybridization) by the 5′ and 3′ homology arms.

“Administering” a nucleic acid, such as a CRISPR system (expressing, e.g., a donor polynucleotide, guide RNA, Cas protein (e.g., Cas9, Cas12a, Cas12d, Cas13, or dCas9)) to a cell comprises transducing, transfecting, electroporating, translocating, fusing, phagocytosing, shooting or ballistic methods, etc., i.e., any means by which a nucleic acid can be transported across a cell membrane.

Generating iPSCs

The iPSCs can be generated by reprogramming somatic cells into pluripotent stem cells. Somatic cells can be induced into forming pluripotent stem cells, for example, by treating them with reprograming factors such as Yamanaka factors, including but not limited to, OCT3, OCT4, SOX2, KLF4, c-MYC, NANOG, and LIN28 (see, e.g., Takahashi et al. (2007) Cell. 131(5):861-872; herein incorporated by reference in its entirety). The types of somatic cells that may be converted into IPSCs include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes. Somatic cells are contacted with reprogramming factors in a combination and quantity sufficient to reprogram the cells to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Methods for “introducing a cell reprogramming factor into somatic cells are not limited in particular, and known procedures can be selected and used as appropriate. For example, when a cell reprogramming factor as described above is introduced into somatic cells of the above-mentioned type in the form of proteins, such methods include ones using protein introducing reagents, fusion proteins with protein transfer domains (PTDs), electroporation, and microinjection. When a cell reprogramming factor as described above is introduced into somatic cells of the above-mentioned type in the form of nucleic acids encoding the cell reprogramming factor, a nucleic acid(s), such as cDNA(s), encoding the cell reprogramming factor can be inserted in an appropriate expression vector comprising a promoter that functions in somatic cells, which then can be introduced into somatic cells by procedures such as infection, lipofection, liposomes, electroporation, calcium phosphate coprecipitation, DEAE-dextran, microinjection, and electroporation. Examples of an “expression vector” include viral vectors, such as lentiviruses, retroviruses, adenoviruses, adeno-associated viruses, and herpes viruses; and expression plasmids for animal cells. For example, retroviral or Sendai virus (SeV) vectors are commonly used to introduce a nucleic acid(s) encoding a cell reprogramming factor as described above into somatic cells.

In some embodiments the IPSCs are derived from somatic cells obtained from normal individuals. In other embodiments the IPSCs are derived from somatic cells obtained from an individual comprising at least one allele encoding an activating mutation associated with a GNAS-associated disorder. The GNAS-associated disorder may include any disorder linked to a mutated GNAS gene comprising an activating mutation (e.g., c.602G>A (p.R201H) or c.601C>T (p.R201C)) and expressing a hyperactive or constitutively active Gsα. Such GNAS-associated disorders include, but are not limited to, fibrous dysplasia (FD), McCune-Albright syndrome (MAS), cancers such as, but not limited to, breast cancer (e.g., breast invasive ductal carcinoma), pancreatic cancer (e.g., pancreatic adenocarcinoma), adrenal cancer, lung cancer (e.g., lung adenocarcinoma, squamous cell lung cancer, small cell lung cancer, and non-small cell lung cancer), colon cancer (e.g., colon adenocarcinoma), rectal cancer (e.g., rectal adenocarcinoma), prostate cancer (e.g., adenocarcinoma), bladder cancer, endometrial cancer, esophageal cancer (e.g., esophageal adenocarcinoma), gastric cancer (e.g., gastric adenocarcinoma), hepatocellular carcinoma, glioblastoma, appendix mucinous adenocarcinoma, cutaneous melanoma, leukemia, and Pseudomyxoma peritonei; and benign tumors such as, but not limited to, intraductal papillary mucinous neoplasms, myxomas, and adenomas. In the case of FD/MAS, creating patient-derived iPSCs may not be feasible because the two main GNAS mutations associated with these diseases do not show germline transmission.

A sample comprising somatic cells for generating iPSCs can be obtained from a subject by any suitable method. The somatic cells may include, without limitation, peripheral blood mononuclear cells, fibroblasts, keratinocytes, epithelial cells, endothelial progenitor cells, mesenchymal stem cells, adipose derived stem cells, leukocytes, hematopoietic stem cells, bone marrow cells, and hepatocytes. The biological sample comprising somatic cells is typically whole blood, buffy coat, peripheral blood mononucleated cells (PBMCS), skin, fat, or a biopsy, but can be any sample from bodily fluids, tissue or cells that contain suitable somatic cells. A biological sample can be obtained from a subject by conventional techniques. For example, blood can be obtained by venipuncture, and solid tissue samples can be obtained by surgical techniques according to methods well known in the art.

### Genome Modification to Introduce Disease-Relevant Genetic Changes

Disease-relevant GNAS mutations can be introduced into the genome of iPSCs or the somatic cells from which they are derived using any method known in the art to produce a cellular model of a GNAS-associated disorder useful for disease modeling and drug screening. In some embodiments, the activating mutation is linked to a GNAS-associated disorder such as fibrous dysplasia, McCune-Albright syndrome, cancer, or benign tumors. For example, a missense mutation can be introduced into a GNAS gene (e.g., c.602G>A (p.R201H) or c.601C>T (p.R201C)) resulting in expression of a hyperactive or constitutively active Gsα. In some cases, one or more additional mutations may be introduced into the GNAS gene. For example, a silent mutation (c.600 C>T) may be introduced into the GNAS gene to facilitate detection of the GNAS transcript.

In certain embodiments, an IPSC is produced comprising a genetically modified GNAS gene comprising a nucleotide sequence selected from the group consisting of SEQ ID NOS:3-8, or a nucleotide sequence having at least about 80-100% sequence identity to a nucleotide sequence selected from the group consisting of SEQ ID NOS:3-8, including any percent identity within this range, such as 81%, 82%, 83%, 84%, 85%, 86%, 87%, 88%, 89%, 90%, 91%, 92%, 93%, 94%, 95%, 96%, 97%, 98%, or 99% sequence identity thereto, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

In some embodiments, one or more activating GNAS mutations and/or other mutations (e.g., p.R201H (c.602G>A), p.R201H (c.602G>A)+silent mutation (c.600 C>T), p.R201C (c.601C>T)+silent mutation (c.600 C>T), or p.R201C (c.601C>T)) are introduced into the genome of an iPSC using engineered nucleases such as, but not limited to, CRISPR/CAS9, meganucleases, zinc finger nucleases (ZFNs), or transcription activator-like effector nucleases (TALENs) for gene editing. See, e.g., CRISPR Gene Editing: Methods and Protocols (edited by Luo, Humana, 2019), Genome Editing and Engineering: From TALENs, ZFNs and CRISPRs to Molecular Surgery (edited by Appasani and Church, Cambridge University Press, 2018); herein incorporated by reference in their entireties.

Genome modification can be performed, for example, using homology directed repair (HDR) with a donor polynucleotide comprising a sequence comprising an intended genome edit (e.g., GNAS activating mutation) flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit is integrated into the genomic DNA by HDR at the genomic target locus recognized (i.e., sufficiently complementary for hybridization) by the 5′ and 3′ homology arms.

In certain embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In a preferred embodiment, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g., 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another, e.g., one may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g., one may be 40% shorter or more, 50% shorter or more, sometimes 60% shorter or more, 70% shorter or more, 80% shorter or more, 90% shorter or more, or 95% shorter or more than the other homology arm.

The donor polynucleotide is used in combination with an RNA-guided nuclease, which is targeted to a particular genomic sequence (i.e., genomic target sequence to be modified) by a guide RNA (gRNA). A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to a target sequence in a gene of interest.

In certain embodiments, the RNA-guided nuclease used for genome modification is a CRISPR system Cas nuclease. Any RNA-guided Cas nuclease capable of catalyzing site-directed cleavage of DNA to allow integration of donor polynucleotides by the HDR mechanism can be used in genome editing, including CRISPR system type I, type II, or type Ill Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, Cas12a (Cpf1), Cas12b (C2c1), Cas12c (C2c3), Cas12d (CasY), Cas12e (CasX), CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC 016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisl (NC_018721); Streptococcus thermophilus (YP_820832), Streptococcus mutans (WP_061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacteriol. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a 5′ protospacer-adjacent motif (PAM) in the DNA at or near the gRNA-binding site.

The genomic target site will typically comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, also known as Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

Cas12b (C2c1) is another class II CRISPR/Cas system RNA-guided nuclease that may be used. C2c1, similarly to Cas9, depends on both a crRNA and tracrRNA for guidance to target sites. For a description of Cas12b, see, e.g., Shmakov et al. (2015) Mol Cell. 60(3):385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided FokI nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

An RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector). In some embodiments, the RNA-guided nuclease and the gRNA are both provided by vectors. Both can be expressed by a single vector or separately on different vectors. The vector(s) encoding the RNA-guided nuclease and gRNA may be included in a CRISPR expression system to target a GNAS gene or other gene of interest.

Codon usage may be optimized to improve production of an RNA-guided nuclease in a particular cell. For example, a nucleic acid encoding an RNA-guided nuclease or reverse transcriptase can be modified to substitute codons having a higher frequency of usage in a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells (e.g., iPSCs or somatic cells from which they are derived), the protein can be transiently, conditionally, or constitutively expressed in the cell.

The gRNAs are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109.

In certain embodiments, a CRISPR system is used to introduce one or more activating mutations linked to a GNAS-associated disease into human iPSCs to produce genetically modified human IPSCs that can be used as a disease model of a GNAS-associated disorder. Exemplary mutations linked to fibrous dysplasia/McCune-Albright syndrome include, without limitation, GNAS mutations such as R201H, R201C, and Q227L, which result in expression of a hyperactive Gsα. See, e.g., Lumbroso et al. (2004) J. Clin. Endocrinol. Metab. 89:2107-2113; and Idowu et al. (2007) Histopathology 50:691-704; herein incorporated by reference).

### Screening Assays

Genetically modified iPSCs comprising an activating GNAS mutation (e.g., p.R201H (c.602G>A), p.R201H (c.602G>A)+silent mutation (c.600 C>T), p.R201C (c.601C>T)+silent mutation (c.600 C>T), or p.R201C (c.601C>T)) expressing a hyperactive Gsα can be subjected to a plurality of candidate agents or other therapeutic intervention. Such activating GNAS mutations, which increase activity of Gsα, have been linked to GNAS-associated disorders, including, but not limited to, fibrous dysplasia (FD), McCune-Albright syndrome (MAS), cancers such as, but not limited to, breast cancer (e.g., breast invasive ductal carcinoma), pancreatic cancer (e.g., pancreatic adenocarcinoma), adrenal cancer, lung cancer (e.g., lung adenocarcinoma, squamous cell lung cancer, small cell lung cancer, and non-small cell lung cancer), colon cancer (e.g., colon adenocarcinoma), rectal cancer (e.g., rectal adenocarcinoma), prostate cancer (e.g., adenocarcinoma), bladder cancer, endometrial cancer, esophageal cancer (e.g., esophageal adenocarcinoma), gastric cancer (e.g., gastric adenocarcinoma), hepatocellular carcinoma, glioblastoma, appendix mucinous adenocarcinoma, cutaneous melanoma, leukemia, and Pseudomyxoma peritonei; and benign tumors such as, but not limited to, intraductal papillary mucinous neoplasms, myxomas, and adenomas. Therefore, inhibitors of the hyperactive Gsα may be useful in treating a GNAS-associated disorder. Accordingly, screening methods for identifying candidate agents that inhibit Gsα for use in treating a GNAS-associated disorder are provided.

A variety of assays may be used for this purpose, and in many embodiments, a candidate agent will be tested in different assays to confirm inhibitory capability as well as binding affinity for mutant and wild-type forms of Gsα, and efficacy in treating a GNAS-associated disorder. For example, biochemical assays may determine the ability of an agent to bind to and inhibit biological activity of Gsα (e.g., GTPase activity, activation of adenylate cyclase).

A “Gsα inhibitor” can be any molecule including, without limitation, a small molecule inhibitor, protein, polypeptide, peptide, fusion protein, nucleic acid, oligonucleotide, peptide nucleic acid, peptoid, antibody or fragment thereof, antibody mimetic, or aptamer that inhibits Gsα biological activity such as GTPase activity and/or ability to activate adenylate cyclase. Inhibition may be complete or partial (i.e., all activity, some activity, or most activity is blocked by an inhibitor). For example, an inhibitor may reduce the activity of Gsα by 10%, 20%, 30%, 40%, 50%, 60%, 70%, 80%, 90%, 100%, or any amount in between as compared to native or control levels. In some embodiments, the inhibitor selectively inhibits the hyperactive Gsα comprising the activating mutation. Preferably, the inhibitor does not fully inhibit the wild-type Gsα.

Inhibitors can be identified by contacting the genetically modified iPSCs with a candidate agent of interest and measuring inhibition of the biological activity of the hyperactive Gsα (e.g., measuring GTPase activity and/or ability to activate adenylate cyclase) by the candidate agent. Any convenient format may be used for the assay, e.g., wells, plates, flasks, etc., preferably a high throughput format, such as multi-well plates.

Inhibition of GTPase activity can be assayed by detecting a decreased rate of production of GDP from GTP in the presence of the candidate agent compared to that in the absence of the candidate agent. For example, the remaining GTP after GTP hydrolysis or the GDP product can be detected by methods known in the art. In an exemplary assay, GTPase activity is measured with a coupled enzyme assay that converts GTP remaining after the GTPase reaction into ATP. The coupled enzyme assay used ADP and a nucleoside diphosphate kinase to catalyze the conversion of GTP to ATP, and a luminescent luciferin/luciferase assay to detect the ATP (see, e.g., Mondal et al. (2015) Assay Drug Dev. Technol. 13(8):444-455, herein incorporated by reference). Alternatively, assays using fluorescently labeled GTP or GDP may be used for measuring GTPase activity. The GTP-bound form or GDP-bound form of the Gsα protein can be measured in cell lysates using, for example, immunoprecipitation, an affinity bead pull-down assay, Western blotting, or an enzyme-linked immunosorbent assay (ELISA). A number of GTPase assay kits are commercially available, such as the GTPase-Glo™ assay from Promega Corporation (Madison, WI), the Transcreener® GDP-GTPase assay from BellBrook Labs LLC (Fitchburg, WI), the Small GTPase Activation Assay from Cell Biolabs, Inc. (San Diego, CA), and the G-LISA Activation Assay from Cytoskeleton, Inc. (Denver, CO). For a description of GTPase assays, see, e.g., Mondal et al. (2015) Assay Drug Dev. Technol. 13(8):444-455; Choudhury et al. (Methods Mol. Biol. (2013) 1043:13-20; Kanie et al. (2018) Bio Protoc. 8(7):e2795), and Zaoui et al. (2020) Bio. Protoc. 10(9):e3609; herein incorporated by reference in their entireties.

Alternatively or additionally, inhibition of activation of adenylate cyclase by the hyperactive Gsα can be assayed by detecting a decreased rate of production of cyclic AMP in the presence of the candidate agent compared to that in the absence of the candidate agent. Adenylate cyclase catalyzes the conversion of ATP to cyclic AMP. The activity of adenylate cyclase may be measured using any method known in the art. For example, the remaining ATP substrate or cyclic AMP product can be detected using radioactive, fluorescent, or bioluminescent assays known in the art. In an exemplary assay, a coupled protein kinase A (PKA)-luciferase enzymatic assay is used to measure intracellular cyclic AMP. ATP and a PKA peptide substrate are added to cell lysates containing cyclic AMP for the assay. The cyclic AMP activates protein kinase A, which uses the ATP to phosphorylate the PKA peptide substrate. Depletion of ATP in the assay by protein kinase A is detected by decreased luminescence in the coupled luciferase reaction. Alternatively, a luminescent luciferin/luciferase assay can be used to directly detect ATP depletion from the activity of the adenylate cyclase. In another example, adenylate cyclase activity is assayed by measuring radiolabeled cyclic AMP generated from [alpha-32P]ATP. A phosphodiesterase inhibitor may be added to assays to prevent the breakdown of cyclic AMP to AMP by cellular phosphodiesterases. In addition, a β-adrenergic receptor agonist (e.g., isoproterenol) may be added to assays to stimulate adenylate cyclase. A number of GTPase assay kits are commercially available, such as the cAMP-Glo™ assay from Promega Corporation (Madison, WI) and the Adenylyl Cyclase Activation FlashPlate assay from PerkinElmer (Waltham, MA). For a description of adenylate cyclase assays, see, e.g., Kumar et al. (2007) Assay Drug Dev. Technol. 5, 237-245, Wiegn et al. (1993) Anal. Biochem. 208(2):217-22, and Israeli et al. (2016) Toxin (Basel) 8(8):243; herein incorporated by reference in their entireties.

The screening assays may also include a binding assay to detect binding of a candidate agent to mutant and wild-type forms of Gsα. Preferably, a candidate agent is identified that selectively binds or exhibits preferential binding to the hyperactive Gsα and does not bind or binds to a lesser extent to the wild-type Gsα. The affinities of binding of a candidate to the mutant and wild-type forms of Gsα can be determined, for example, by surface plasmon resonance (SPR), isothermal titration calorimetry (ITC), or radioisotopic or spectroscopic techniques. In some cases, the candidate agent may be labeled directly or indirectly to provide a detectable signal. Various labels may be used including, without limitation, radioisotopes, fluorescers, chemiluminescers, enzymes, specific binding molecules, particles (e.g., magnetic particles), and the like. Candidate agents may be further screened for binding to a G protein-coupled receptor (GPCR) that activates the hyperactive Gsα.

Assays may further include suitable controls (e.g., a sample comprising the Gsα in the absence of the test agent). Generally, a plurality of assay mixtures is run in parallel with different agent concentrations to obtain a differential response to the various concentrations. Typically, one of these concentrations serves as a negative control, i.e., at zero concentration or below the level of detection.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

A variety of different test agents may be screened. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit significant cytotoxic activity are considered candidate agents.

In some embodiments, the test agent is an antibody that specifically binds to and inhibits biological activity of a hyperactive Gsα. Any type of antibody may be screened for the ability to inhibit a hyperactive Gsα by the methods described herein, including polyclonal antibodies, monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

In other embodiments, the test agent is an aptamer that specifically binds to and inhibits biological activity of a hyperactive Gsα. Aptamers may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Aptamers Selected by Cell-SELEX for Theranostics (W. Tan, X. Fang eds., Springer, 2015), Cox et al. (2001) Bioorg. Med. Chem. 9(10):2525-2531; Cox et al. (2002) Nucleic Acids Res. 30(20): e108, Kenan et al. (1999) Methods Mol. Biol. 118:217-231; Platella et al. (2016) Biochim. Biophys. Acta Nov 16 pii: S0304-4165(16)30447-0, and Lyu et al. (2016) Theranostics 6(9):1440-1452; herein incorporated by reference in their entireties.

In yet other embodiments, the test agent is an antibody mimetic that specifically binds to and inhibits biological activity of a hyperactive Gsα. Any type of antibody mimetic may be used as an inhibitor, including, but not limited to, affibody molecules (Nygren (2008) FEBS J. 275 (11):2668-2676), affilins (Ebersbach et al. (2007) J. Mol. Biol. 372 (1):172-185), affimers (Johnson et al. (2012) Anal. Chem. 84 (15):6553-6560), affitins (Krehenbrink et al. (2008) J. Mol. Biol. 383 (5):1058-1068), alphabodies (Desmet et al. (2014) Nature Communications 5:5237), anticalins (Skerra (2008) FEBS J. 275 (11):2677-2683), avimers (Silverman et al. (2005) Nat. Biotechnol. 23 (12):1556-1561), darpins (Stumpp et al. (2008) Drug Discov. Today 13 (15-16):695-701), fynomers (Grabulovski et al. (2007) J. Biol. Chem. 282 (5):3196-3204), and monobodies (Koide et al. (2007) Methods Mol. Biol. 352:95-109); herein incorporated by reference in their entireties.

In yet other embodiments, the test agent is peptoid that specifically binds to and inhibits biological activity of a hyperactive Gsα. The term “peptoid” refers to an oligomer comprising two or more N-substituted glycine residues. The side chain of each residue in a peptoid is connected to the amide nitrogen of the peptoid backbone, instead of the α-carbon as in peptides. Libraries of short peptoid oligomers may be screened for binding and inhibition of a hyperactive Gsα. For a description of methods of synthesizing peptoids and peptoid combinatorial libraries, see, e.g., Cardenal Design, Synthesis and Evaluation of Highly Functionalized Peptoids as Antitumor Peptidomimetics (Beitrage Zur Organischen Synthese, Logos Verlag, 2014), Zuckermann et al. (2011) Biopolymers 96(5):545-55, Clapperton et al. (2022) ACS Polym Au. 2(6):417-429, Yoo et al. (2008) Curr. Opin. Chem. Biol. 12(6):714-21, Webster et al. (2018) Chemistry 24(30):7560-7573, Singh et al. (2016) Biopolymers 106(5):673-84, Zuckermann et al. (2009) Curr Opin Mol Ther. 11(3):299-307, Herlan et al. (2021) Chem. Commun. (Camb). 57(85):11131-11152; herein incorporated by reference in their entireties.

Candidate agents are screened for inhibition of Gsα biological activity by adding the agent to at least one and usually a plurality of iPSCs under one or in a plurality of environmental conditions. The response to the agent is measured, preferably normalized, and the resulting screening results are evaluated by comparison to reference screening results, e.g., with iPSCs in the absence of the candidate agent, iPSCs having a wild-type GNAS gene or a GNAS gene with other mutations of interest in the presence and absence of the candidate agent, and the like. The reference screening results may also include results obtained with iPSCs in the presence and absence of other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on Gsα activity or the iPSCs. Thus, preferred formulations consist essentially of a biologically active test compound and a physiologically acceptable carrier, e.g., water, ethanol, DMSO, etc. However, if a compound is liquid without a solvent, the formulation may consist essentially of the compound itself.

In some embodiments, a test compound identified as an inhibitor of a hyperactive Gsα in iPSC-based assays is further tested for its efficacy in treating a GNAS-associated disorder in vivo, e.g., in an animal such as an animal model of a GNAS-associated disorder. For example, an agent that inhibits biological activity of a hyperactive Gsα, identified as described herein, can be used in an animal model to determine the efficacy, toxicity, or side effects of treatment with such an agent. Alternatively, an agent identified, as described herein, can be used in an animal model to determine the mechanism of action of such an agent. Monitoring the efficacy of agents (e.g., drugs) on a GNAS-associated disorder can be applied not only in basic drug screening, but also in clinical trials. Furthermore, this disclosure pertains to uses of novel agents identified by the above-described screening assays for treatment of a GNAS-associated disorder.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of screening for an agent that inhibits activity of a hyperactive stimulatory guanine nucleotide binding protein alpha subunit (Gsα) for use in treating a guanine nucleotide-binding protein alpha, stimulatory (GNAS)-associated disorder, the method comprising:
providing an induced pluripotent stem cell (iPSC) comprising a genetically modified GNAS gene comprising an activating mutation, wherein the genetically modified GNAS gene comprises a coding sequence encoding a hyperactive Gsα;
contacting the iPSC with a candidate agent; and
measuring inhibition of the hyperactive Gsα by the candidate agent.

2. The method of aspect 1, further comprising: contacting an iPSC comprising an unmodified GNAS gene encoding a wild-type Gsα with the candidate agent;
measuring inhibition of activity of the wild-type Gsα by the candidate agent;
comparing levels of inhibition of the wild-type Gsα and the hyperactive Gsα, wherein increased inhibition of the hyperactive Gsα compared to the wild-type Gsα indicates that the candidate agent selectively inhibits the hyperactive Gsα.

3. The method of aspect 1 or 2, wherein the GNAS-associated disorder is fibrous dysplasia, McCune-Albright syndrome, or cancer.

4. The method of aspect 3, wherein the cancer is breast cancer, pancreatic cancer, or adrenal cancer.

5. The method of any one of aspects 1-4, wherein the activating mutation is linked to fibrous dysplasia, McCune-Albright syndrome, or cancer.

6. The method of aspect 5, wherein the hyperactive Gsα comprises a substitution of histidine or cysteine at amino acid position 201, wherein numbering of amino acid positions is relative to the reference amino acid sequence of SEQ ID NO:1.

7. The method of aspect 5, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

8. The method of aspect 7, wherein the genetically modified GNAS gene further comprises a substitution of thymine at nucleotide position 600, wherein numbering of nucleotide positions is relative to the reference nucleotide sequence of SEQ ID NO:2.

9. The method of any one of aspects 1-8, wherein a CRISPR system is used to genetically modify the genome of the iPSC to introduce the activating mutation into the GNAS gene, wherein a hyperactive Gsα is expressed in the iPSC.

10. The method of aspect 9, wherein the CRISPR system comprises a guide RNA (gRNA) capable of hybridizing to a target site in the GNAS gene.

11. The method of any one of aspects 1-10, wherein said measuring inhibition of the Gsα comprises measuring inhibition of activation of adenylate cyclase activity by the hyperactive Gsα.

12. The method of aspect 11, wherein said measuring inhibition of activation of adenylate cyclase activity comprises measuring levels of intracellular cyclic adenosine monophosphate (cAMP) in presence and absence of the candidate agent, wherein reduced levels of cAMP in the presence of the candidate agent compared to in the absence of the candidate agent indicate that the candidate agent inhibits activation of adenylate cyclase activity by the hyperactive Gsα.

13. The method of any one of aspects 1-12, wherein said measuring inhibition of the Gsα comprises measuring inhibition of GTPase activity of the Gsα.

14. The method of any one of aspects 1-13, further comprising measuring binding of the candidate agent to the hyperactive Gsα.

15. The method of any one of aspects 1-14, further comprising measuring binding of the candidate agent to a G protein-coupled receptor (GPCR) that activates the hyperactive Gsα.

16. The method of any one of aspects 1-15, wherein the candidate agent does not fully inhibit the wild-type Gsα.

17. The method of any one of aspects 1-16, wherein the agent is a small molecule, a drug, a peptide, a protein, an aptamer, a peptoid, an antibody, or an antibody mimetic.

18. The method of any one of aspects 1-17, wherein the iPSC is heterozygous for the genetically modified GNAS gene comprising the activating mutation.

19. The method of any one of aspects 1-18, further comprising: contacting the iPSC with a β-adrenergic receptor agonist; and measuring inhibition of the hyperactive Gsα by the candidate agent in presence of the β-adrenergic receptor agonist.

20. The method of aspect 19, wherein the β-adrenergic receptor agonist is isoproterenol.

21. The method of any one of aspects 1-20, further comprising:
contacting the iPSC with a phosphodiesterase inhibitor; and
measuring inhibition of the hyperactive Gsα by the candidate agent in presence of the phosphodiesterase inhibitor.

22. The method of any one of aspects 1-21, wherein the genetically modified GNAS gene comprises a nucleotide sequence selected from the group consisting of SEQ ID NOS:3-8.

23. The method of any one of aspects 1-21, wherein the genetically modified GNAS gene comprises a nucleotide sequence having at least 90% identity to a sequence selected from the group consisting of SEQ ID NOS:3-8, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

24. A composition comprising:
an induced pluripotent stem cell (iPSC) comprising a genetically modified GNAS gene comprising an activating mutation, wherein the genetically modified GNAS gene comprises a coding sequence encoding a hyperactive stimulatory guanine nucleotide binding protein alpha subunit (Gsα).

25. The composition of aspect 24, wherein the activating mutation is linked to fibrous dysplasia, McCune-Albright syndrome, or cancer.

26. The composition of aspect 25, wherein the hyperactive Gsα comprises a substitution of histidine or cysteine at amino acid position 201, wherein numbering of amino acid positions is relative to the reference amino acid sequence of SEQ ID NO:1.

27. The composition of aspect 25, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

28. The composition of aspect 27, wherein the genetically modified GNAS gene further comprises a substitution of thymine at nucleotide position 600, wherein numbering of nucleotide positions is relative to the reference nucleotide sequence of SEQ ID NO:2.

29. The composition of any one of aspects 24-28, further comprising a β-adrenergic receptor agonist.

30. The composition of aspect 29, wherein the β-adrenergic receptor agonist is isoproterenol.

31. The composition of any one of aspects 24-30, further comprising a phosphodiesterase inhibitor.

32. The composition of any one of aspects 24-31, wherein the genetically modified GNAS gene comprises a nucleotide sequence selected from the group consisting of SEQ ID NOS:3-8.

33. The composition of any one of aspects 24-31, wherein the genetically modified GNAS gene comprises a nucleotide sequence having at least 90% identity to a sequence selected from the group consisting of SEQ ID NOS:3-8, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

Human iPSCs Containing a GNAS R201H Mutation Linked to FD/MAS

### Introduction

FD, accounts for 2.5% of all bone lesions and 7% of benign skeletal dysplasias (45), with craniofacial and long bones being major sites (46). Although the genetic mutations that lead to FD are known and are located at the GNAS locus, medical treatments for this disfiguring disorder are sorely lacking (47). FD can occur in isolation or as part of MAS, a mosaic genetic condition characterized by polyostotic FD (i.e., affecting multiple bones), café-au-lait skin hyperpigmentation, precocious puberty, endocrinopathies (e.g., Cushing's disease), hyperthyroidism, acromegaly, and solid organ malignancies (48-50). Although MAS affects numerous tissues, FD can develop independently of these other conditions and is arguably the most significant manifestation because there are no effective pharmacologic treatments for the bone complications (51) (Error! Reference source not found.). Many FD lesions are associated with pain and fractures. The mainstay of FD treatment remains watchful waiting and judicious surgical resection, which is often complex due to the size and location of the affected bones (51).

FD/MAS is commonly caused by a somatic activating mutation in the guanine nucleotide-binding protein alpha, stimulatory (GNAS) gene, which encodes the a subunit of the stimulatory guanine nucleotide binding protein (Gₛα) that increases production of intracellular cAMP. The most common variant, c.602G>A p.R201H, causes constitutive activation of Gₛα by inhibiting the GTP hydrolase activity of Gₛα, leading to persistently elevated intracellular cAMP and increased downstream cAMP pathway activity.

FD lesions show significant histologic and radiologic variability (47; 52; 53), but share a characteristic spindle-like fibroblastic-appearing cellular infiltrate with traits of immature osteoblasts (3; 52; 54; 55). The origin, identity, and function of these cells are unknown. FD bone lesions contain prolific amounts of postnatal trabecular bone, and the observation that FD bone can grow with growth hormone hypersecretion (46), even in adults, suggests that anabolic bone cells are present into adulthood. Furthermore, the obligate genetic mosaicism in FD (56; 57) allows us to use the GNAS mutation as a natural marker to identify the relevant cells in the niche that contribute to the dramatic anabolic effect. We use these features in our studies to identify the potential cells and mechanisms that could be harnessed for postnatal bone growth and repair. Our preliminary data describe our rationale, key tools, and feasibility for this study.

### Activated Gₛ Signaling in Osteoblastic Cells Induces Skeletal and Craniofacial Bone Formation

Until recently, direct genetic targeting of GNAS was limited by the complexity of the locus and technical limitations for introducing single point mutations. When we first started in 2005, the direct role of activated Gₛ signaling in osteoblasts had not been elucidated, and models with reversible activation of a single GPCR pathway in a tissue-specific manner were not available. To circumvent this, we used an engineered receptor “Rs1” (58; 59) that no longer responded to endogenous hormones but retained strong basal cAMP/Gₛ signaling activity and could be activated by synthetic ligands. This allowed us to study both constitutive receptor and ligand-mediated activation of Gₛ-GPCR signaling (3; 60).

Coll(2.3)⁺/Rs1⁺ mice expressing Rs1 in osteoblastic cells with a Collagen 1 a1 2.3 kb promoter [Coll(2.3)] had dramatically increased bone formation with loss of marrow space and cortical bone (3; 61) (Error! Reference source not found.) and changes in fracture repair (31) and hematopoiesis (28). Calvaria showed similar changes (10) (Error! Reference source not found.A, 2B, 2D). These features resemble human FD (Error! Reference source not found.C). Histology (3) and fluorescent activated cell sorting (FACS) (28) analyses showed increases in immature osteoblasts, suggesting that activating the cAMP pathway blocks osteoblast maturation and leads to accumulation of immature precursors. The FD bone also shifted the distribution of cell types: stromal cells were increased (28) and adipocytes decreased (62), suggesting that activating Gₛ-GPCR activity in osteoblasts affects the plasticity of precursors in the bone niche.

### Postnatal Bone Responds to Activation and De-Activation of the Gₛ Pathway

Human FD shows age dependence, where the majority of FD bone formation typically occurs before the third decade of life. Although both human FD (51) and Coll(2.3)⁺/Rs1⁺ FD-like (12) bone lesions show age-related attenuation of bone formation, ligand activation of these mice could induce huge amounts of FD bone formation (60) postnatally. Surprisingly, stopping excess Gₛ signaling reversed the FD bone lesions (Error! Reference source not found.E) (12). This provides proof-of-concept that pharmacologic reversal of Gₛ over-activation may be a treatment strategy for FD and may act by inhibiting the differentiation of osteogenic precursors.

Human iPSCs are Valuable Tools for Understanding Plasticity, and Generation of Human FD/MAS iPSCs

iPSCs are powerful tools for studying disease as they can form tissues of all germ layers. We have extensive experience using iPSCs to study human diseases (63-69). The mosaic nature of FD/MAS meant that standard approaches for creating patient-derived iPSCs were not feasible: blood monocytes rarely carry the FD/MAS mutation and are not used in clinical genetics, and only melanocytes (café au lait spots) are affected, making skin samples unsuitable. Our attempts to make iPSCs from bone marrow cells were unsuccessful (not shown). Because the two main GNAS^{R201C/H} mutations in FD do not show germline transmission, we used CRISPR/Cas9 genetic recombineering (70) on well-established, control iPSC lines [WTc11, Corielle #GM25256 and 1323 (67)] to introduce the GNAS^{R201H} mutation into the endogenous locus. The isogenic GNAS^{R201H} human iPSCs are fully characterized, express pluripotency markers, form all three germ layers in vitro (in embryoid bodies) and in vivo (in teratomas), and carry GNAS^{R201H} in the heterozygous state (data not shown).

GNAS^{R201H} iPSCs had increases in cAMP levels, present from baseline through increasing doses of forskolin treatment (Error! Reference source not found.A). This is consistent with the expected increased activity of the genetic mutation and similar in magnitude to what has been reported in the literature (71). Testing of two other cell clones derived by the same technique confirmed our findings of increased cAMP production. Western blot analysis (Error! Reference source not found.B) showed that increased cAMP response element-binding protein (CREB) and cAMP-dependent activating transcription factor 1 (ATF1) phosphorylation could be detected, indicating that the increased cAMP activates downstream signaling pathways. Finally, bulk RNAseq using ENRICHR pathway analysis (72) confirmed increased cAMP pathway activity. These GNAS^{R201H} iPSCs are a unique tool for studying human FD and GNAS function.

### Example 2

Identification of Compound Scaffolds that Show Selective Reduction of cAMP Production by the Gsα R201H Protein

Through a completed collaboration with Atomwise (73), we screened large libraries of compounds for molecules that may selectively bind Gₛα^{R201H} (Error! Reference source not found.A). The structures were computationally screened to identify molecules that preferentially fit the Gₛα^{R201H} structure, but not the WT. The criteria for probable preferential binding to Gₛα^{R201H} were met by 71 compounds and scaffolds. These are available to us for research.

Initial screens with a single dose (50 μM) in our GNAS iPSCs identified several compounds with potential as molecular tools (Error! Reference source not found.B). We found candidates that inhibited Gₛα^{R201H} but not WT (#46, 47, 43, 2). Others decreased cAMP in Gₛα^{R201H} and WT (#50, 51), and some increased cAMP production (#32,60). While not desirable for treating FD/MAS or GNAS hyperactivation, these may be useful for treating diseases of GNAS loss of function (e.g., Albright's hereditary osteodystrophy). One key limitation of this initial screen was that we assessed each compound for their ability to suppress basal cAMP production, but that these absolute levels were low and showed variability. More recent data using our iPSCs stimulated with the β-adrenergic receptor agonist, isoproterenol, indicates that a greater discrimination of cAMP levels may be possible (Error! Reference source not found.C).

Our preferred compounds should not fully inhibit Gₛα, since Gₛα is widely expressed, has no redundancy, and full suppression is likely toxic. Our screen was done with a phosphodiesterase inhibitor and without forskolin, suggesting our findings result from activity on Gₛα and not downstream factors. Thus, our assay may detect changes in cAMP, and compounds that alter Gₛαactivity can be found.

### REFERENCES CITED

1. Fredriksson, R., Lagerstrom, M. C., Lundin, L. G., and Schioth, H. B. (2003). The G-protein-coupled receptors in the human genome form five main families. Phylogenetic analysis, paralogon groups, and fingerprints. Mol Pharmacol 63(6), 1256-1272.
2. Karchin, R., Karplus, K., and Haussler, D. (2002). Classifying G-protein coupled receptors with support vector machines. Bioinformatics 18(1), 147-159.

3. Hsiao, E. C., Boudignon, B. M., Chang, W. C., Bencsik, M., Peng, J., Nguyen, T. D., Manalac, C., Halloran, B. P., Conklin, B. R., and Nissenson, R. A. (2008). Osteoblast expression of an engineered Gs-coupled receptor dramatically increases bone mass. Proc Natl Acad Sci USA 105(4), 1209-1214.
4. National Osteoporosis Foundation (2002). America's Bone Health: The State of Osteoporosis and Low Bone Mass in Our Nation (Washington, D. C., National Osteoporosis Foundation).
5. Brink, C. B., Harvey, B. H., Bodenstein, J., Venter, D. P., and Oliver, D. W. (2004). Recent advances in drug action and therapeutics: relevance of novel concepts in G-protein-coupled receptor and signal transduction pharmacology. Br J Clin Pharmacol 57(4), 373-387.
6. Boyce, A. M., Chong, W. H., Yao, J., Gafni, R. I., Kelly, M. H., Chamberlain, C. E., Bassim, C., Cherman, N., Ellsworth, M., Kasa-Vubu, J. Z., Farley, F. A., Molinolo, A. A., Bhattacharyya, N., and Collins, M. T. (2012). Denosumab treatment for fibrous dysplasia. Journal of bone and mineral research: the official journal of the American Society for Bone and Mineral Research 27(7), 1462-1470.
7. Seifert, R., and Wenzel-Seifert, K. (2002). Constitutive activity of G-protein-coupled receptors: cause of disease and common property of wild-type receptors. Naunyn Schmiedebergs Arch Pharmacol 366(5), 381-416.
8. Perez, D. M. (2002). Polymorphic G-protein-coupled receptors and associated diseases. Receptors Channels 8(1), 57-64.
9. Malbon, C. C. (2005). G proteins in development. Nat Rev Mol Cell Biol 6(9), 689-701.
10. Wattanachanya, L., Wang, L., Millard, S. M., Lu, W. D., O'Carroll, D., Hsiao, E. C., Conklin, B. R., and Nissenson, R. A. (2015). Assessing the osteoblast transcriptome in a model of enhanced bone formation due to constitutive Gs-G protein signaling in osteoblasts. Exp Cell Res 333(2), 289-302.
11. Gether, U. (2000). Uncovering molecular mechanisms involved in activation of G protein-coupled receptors. Endocr Rev 21(1), 90-113.
12. Hsiao, E. C., Boudignon, B. M., Halloran, B. P., Nissenson, R. A., and Conklin, B. R. (2010). Gs G protein-coupled receptor signaling in osteoblasts elicits age-dependent effects on bone formation. J Bone Miner Res 25(3), 584-593.
13. Juppner, H., Abou-Samra, A. B., Freeman, M., Kong, X. F., Schipani, E., Richards, J., Kolakowski, L. F., Jr., Hock, J., Potts, J. T., Jr., Kronenberg, H. M., and Segre, G. V. (1991). A G protein-linked receptor for parathyroid hormone and parathyroid hormone-related peptide. Science 254(5034), 1024-1026.
14. Kasperk, C. H., Borcsok, I., Schairer, H. U., Schneider, U., Nawroth, P. P., Niethard, F. U., and Ziegler, R. (1997). Endothelin-1 is a potent regulator of human bone cell metabolism in vitro. Calcif Tissue Int 60(4), 368-374.
15. Suzawa, T., Miyaura, C., Inada, M., Maruyama, T., Sugimoto, Y., Ushikubi, F., Ichikawa, A., Narumiya, S., and Suda, T. (2000). The role of prostaglandin E receptor subtypes (EP1, EP2, EP3, and EP4) in bone resorption: an analysis using specific agonists for the respective EPs. Endocrinology 141(4), 1554-1559.
16. Moore, R. E., Smith, C. K., 2nd, Bailey, C. S., Voelkel, E. F., and Tashjian, A. H., Jr. (1993). Characterization of beta-adrenergic receptors on rat and human osteoblast-like cells and demonstration that beta-receptor agonists can stimulate bone resorption in organ culture. Bone Miner 23(3), 301-315.
17. Togari, A., Arai, M., Mizutani, S., Mizutani, S., Koshihara, Y., and Nagatsu, T. (1997). Expression of mRNAs for neuropeptide receptors and beta-adrenergic receptors in human osteoblasts and human osteogenic sarcoma cells. Neurosci Lett 233(2-3), 125-128.
18. Abe, E., Marians, R. C., Yu, W., Wu, X. B., Ando, T., Li, Y., Iqbal, J., Eldeiry, L., Rajendren, G., Blair, H. C., Davies, T. F., and Zaidi, M. (2003). TSH is a negative regulator of skeletal remodeling. Cell 115(2), 151-162.
19. Vilarino-Guell, C., Miles, L. J., Duncan, E. L., Ralston, S. H., Compston, J. E., Cooper, C., Langdahl, B. L., Maclelland, A., Pols, H. A., Reid, D. M., Uitterlinden, A. G., Steer, C. D., Tobias, J. H., Wass, J. A., and Brown, M. A. (2007). PTHR1 Polymorphisms Influence BMD Variation through Effects on the Growing Skeleton. Calcif Tissue Int.
20. Calvi, L. M., Sims, N. A., Hunzelman, J. L., Knight, M. C., Giovannetti, A., Saxton, J. M., Kronenberg, H. M., Baron, R., and Schipani, E. (2001). Activated parathyroid hormone/parathyroid hormone-related protein receptor in osteoblastic cells differentially affects cortical and trabecular bone. J Clin Invest 107(3), 277-286.
21. Rixon, R. H., Whitfield, J. F., Gagnon, L., Isaacs, R. J., Maclean, S., Chakravarthy, B., Durkin, J. P., Neugebauer, W., Ross, V., Sung, W., and et al. (1994). Parathyroid hormone fragments may stimulate bone growth in ovariectomized rats by activating adenylyl cyclase. J Bone Miner Res 9(8), 1179-1189.
22. Armamento-Villareal, R., Ziambaras, K., Abbasi-Jarhomi, S. H., Dimarogonas, A., Halstead, L., Fausto, A., Avioli, L. V., and Civitelli, R. (1997). An intact N terminus is required for the anabolic action of parathyroid hormone on adult female rats. J Bone Miner Res 12(3), 384-392.
23. Hilliker, S., Wergedal, J. E., Gruber, H. E., Bettica, P., and Baylink, D. J. (1996). Truncation of the amino terminus of PTH alters its anabolic activity on bone in vivo. Bone 19(5), 469-477.
24. Whitfield, J. F., Morley, P., Willick, G. E., Ross, V., Barbier, J. R., Isaacs, R. J., and Ohannessian-Barry, L. (1996). Stimulation of the growth of femoral trabecular bone in ovariectomized rats by the novel parathyroid hormone fragment, hPTH-(1-31)NH2 (Ostabolin). Calcif Tissue Int 58(2), 81-87.
25. Isogai, Y., Akatsu, T., Ishizuya, T., Yamaguchi, A., Hori, M., Takahashi, N., and Suda, T. (1996). Parathyroid hormone regulates osteoblast differentiation positively or negatively depending on the differentiation stages. Journal of bone and mineral research: the official journal of the American Society for Bone and Mineral Research 11(10), 1384-1393.
26. Dobnig, H., and Turner, R. T. (1997). The effects of programmed administration of human parathyroid hormone fragment (1-34) on bone histomorphometry and serum chemistry in rats. Endocrinology 138(11), 4607-4612.
27. Neer, R. M., Arnaud, C. D., Zanchetta, J. R., Prince, R., Gaich, G. A., Reginster, J. Y., Hodsman, A. B., Eriksen, E. F., Ish-Shalom, S., Genant, H. K., Wang, O., and Mitlak, B. H. (2001). Effect of parathyroid hormone (1-34) on fractures and bone mineral density in postmenopausal women with osteoporosis. N Engl J Med 344(19), 1434-1441.
28. Schepers, K., Hsiao, E. C., Garg, T., Scott, M. J., and Passegue, E. (2012). Activated Gs signaling in osteoblastic cells alters the hematopoietic stem cell niche in mice. Blood 120(17), 3425-3435.
29. Wu, J. Y., Scadden, D. T., and Kronenberg, H. M. (2009). Role of the osteoblast lineage in the bone marrow hematopoietic niches. J Bone Miner Res 24(5), 759-764.
30. Calvi, L. M., Adams, G. B., Weibrecht, K. W., Weber, J. M., Olson, D. P., Knight, M. C., Martin, R. P., Schipani, E., Divieti, P., Bringhurst, F. R., Milner, L. A., Kronenberg, H. M., and Scadden, D. T. (2003). Osteoblastic cells regulate the haematopoietic stem cell niche. Nature 425(6960), 841-846.
31. Wang, L., Hsiao, E. C., Lieu, S., Scott, M., O'Carroll, D., Urrutia, A., Conklin, B. R., Colnot, C., and Nissenson, R. A. (2015). Loss of Gᵢ G-Protein-Coupled Receptor Signaling in Osteoblasts Accelerates Bone Fracture Healing. J Bone Miner Res 30(10), 1896-1904.
32. Alkhiary, Y. M., Gerstenfeld, L. C., Krall, E., Westmore, M., Sato, M., Mitlak, B. H., and Einhorn, T. A. (2005). Enhancement of experimental fracture-healing by systemic administration of recombinant human parathyroid hormone (PTH 1-34). J Bone Joint Surg Am 87(4), 731-741.
33. Jilka, R. L. (2007). Molecular and cellular mechanisms of the anabolic effect of intermittent PTH. Bone 40(6), 1434-1446.
34. van der Horst, G., Farih-Sips, H., Lowik, C. W., and Karperien, M. (2005). Multiple mechanisms are involved in inhibition of osteoblast differentiation by PTHrP and PTH in KS483 Cells. J Bone Miner Res 20(12), 2233-2244.
35. Regard, J. B., Zhong, Z., Williams, B. O., and Yang, Y. (2012). Wnt signaling in bone development and disease: making stronger bone with Wnts. Cold Spring Harb Perspect Biol 4(12).
36. Khan, S. K., Yadav, P. S., Elliott, G., Hu, D. Z., Xu, R., and Yang, Y. (2018). Induced Gnas(R201H) expression from the endogenous Gnas locus causes fibrous dysplasia by up-regulating Wnt/beta-catenin signaling. Proc Natl Acad Sci USA 115(3), E418-E427.
37. Pan, A., Chang, L., Nguyen, A., and James, A. W. (2013). A review of hedgehog signaling in cranial bone development. Frontiers in physiology 4, 61.
38. Yu, F. X., Zhao, B., Panupinthu, N., Jewell, J. L., Lian, I., Wang, L. H., Zhao, J., Yuan, H., Tumaneng, K., Li, H., Fu, X. D., Mills, G. B., and Guan, K. L. (2012). Regulation of the Hippo-YAP pathway by G-protein-coupled receptor signaling. Cell 150(4), 780-791.
39. Abou-Samra, A. B., Juppner, H., Force, T., Freeman, M. W., Kong, X. F., Schipani, E., Urena, P., Richards, J., Bonventre, J. V., Potts, J. T., Jr., Kronenberg, H. M., and Segre, G. V. (1992). Expression cloning of a common receptor for parathyroid hormone and parathyroid hormone-related peptide from rat osteoblast-like cells: a single receptor stimulates intracellular accumulation of both cAMP and inositol trisphosphates and increases intracellular free calcium. Proc Natl Acad Sci USA 89(7), 2732-2736.
40. Bringhurst, F. R., Juppner, H., Guo, J., Urena, P., Potts, J. T., Jr., Kronenberg, H. M., Abou-Samra, A. B., and Segre, G. V. (1993). Cloned, stably expressed parathyroid hormone (PTH)/PTH-related peptide receptors activate multiple messenger signals and biological responses in LLC-PK1 kidney cells. Endocrinology 132(5), 2090-2098.
41. Gensure, R. C., Gardella, T. J., and Juppner, H. (2005). Parathyroid hormone and parathyroid hormone-related peptide, and their receptors. Biochem Biophys Res Commun 328(3), 666-678.
42. Regard, J. B., Cherman, N., Palmer, D., Kuznetsov, S. A., Celi, F. S., Guettier, J. M., Chen, M., Bhattacharyya, N., Wess, J., Coughlin, S. R., Weinstein, L. S., Collins, M. T., Robey, P. G., and Yang, Y. (2011). Wnt/beta-catenin signaling is differentially regulated by Galpha proteins and contributes to fibrous dysplasia. Proc Natl Acad Sci USA 108(50), 20101-20106.
43. Remoli, C., Michienzi, S., Sacchetti, B., Consiglio, A. D., Cersosimo, S., Spica, E., Robey, P. G., Holmbeck, K., Cumano, A., Boyde, A., Davis, G., Saggio, I., Riminucci, M., and Bianco, P. (2015). Osteoblast-specific expression of the fibrous dysplasia (FD)-causing mutation Gsalpha(R201C) produces a high bone mass phenotype but does not reproduce FD in the mouse. Journal of bone and mineral research: the official journal of the American Society for Bone and Mineral Research 30(6), 1030-1043.
44. Baldridge, D., Shchelochkov, O., Kelley, B., and Lee, B. (2010). Signaling pathways in human skeletal dysplasias. Annual review of genomics and human genetics 11, 189-217.
45. Ricalde, P., and Horswell, B. B. (2001). Craniofacial fibrous dysplasia of the fronto-orbital region: a case series and literature review. J Oral Maxillofac Surg 59(2), 157-167; discussion 167-158.
46. Lee, J. S., FitzGibbon, E. J., Chen, Y. R., Kim, H. J., Lustig, L. R., Akintoye, S. O., Collins, M. T., and Kaban, L. B. (2012). Clinical guidelines for the management of craniofacial fibrous dysplasia. Orphanet journal of rare diseases 7 Suppl 1, S2.
47. Chapurlat, R. D., and Orcel, P. (2008). Fibrous dysplasia of bone and McCune-Albright syndrome. Best practice & research Clinical rheumatology 22(1), 55-69.
48. Turan, S., and Bastepe, M. (2015). GNAS Spectrum of Disorders. Curr Osteoporos Rep 13(3), 146-158.
49. Collins, M. T., Singer, F. R., and Eugster, E. (2012). McCune-Albright syndrome and the extraskeletal manifestations of fibrous dysplasia. Orphanet J Rare Dis 7 Suppl 1, S4.
50. Dumitrescu, C. E., and Collins, M. T. (2008). McCune-Albright syndrome. Orphanet journal of rare diseases 3, 12.
51. Javaid, M. K., Boyce, A., Appelman-Dijkstra, N., Ong, J., Defabianis, P., et al. (2019). Best practice management guidelines for fibrous dysplasia/McCune-Albright syndrome: a consensus statement from the FD/MAS international consortium. Orphanet J Rare Dis 14(1), 139.
52. Collins, M. T. (2006). Spectrum and natural history of fibrous dysplasia of bone. J Bone Miner Res 21 Suppl 2, P99-P104.
53. Kushchayeva, Y. S., Kushchayev, S. V., Glushko, T. Y., Tella, S. H., Teytelboym, O. M., Collins, M. T., and Boyce, A. M. (2018). Fibrous dysplasia for radiologists: beyond ground glass bone matrix. Insights into imaging 9(6), 1035-1056.
54. Harris, W. H., Dudley, H. R., Jr., and Barry, R. J. (1962). The natural history of fibrous dysplasia. An orthopaedic, pathological, and roentgenographic study. The Journal of bone and joint surgery American volume 44-A, 207-233.
55. Marie, P. J., de Pollak, C., Chanson, P., and Lomri, A. (1997). Increased proliferation of osteoblastic cells expressing the activating Gₛ alpha mutation in monostotic and polyostotic fibrous dysplasia. Am J Pathol 150(3), 1059-1069.
56. Happle, R. (1986). The McCune-Albright syndrome: a lethal gene surviving by mosaicism. Clinical genetics 29(4), 321-324.
57. Bianco, P., Kuznetsov, S. A., Riminucci, M., Fisher, L. W., Spiegel, A. M., and Robey, P. G. (1998). Reproduction of human fibrous dysplasia of bone in immunocompromised mice by transplanted mosaics of normal and Gsalpha-mutated skeletal progenitor cells. J Clin Invest 101(8), 1737-1744.
58. Conklin, B. R., Hsiao, E. C., Claeysen, S., Dumuis, A., Srinivasan, S., Forsayeth, J. R., Guettier, J. M., Chang, W. C., Pei, Y., McCarthy, K. D., Nissenson, R. A., Wess, J., Bockaert, J., and Roth, B. L. (2008). Engineering GPCR signaling pathways with RASSLs. Nat Methods 5(8), 673-678.
59. Chang, W. C., Ng, J. K., Nguyen, T., Pellissier, L., Claeysen, S., Hsiao, E. C., and Conklin, B. R. (2007). Modifying ligand-induced and constitutive signaling of the human 5-HT4 receptor. PLoS One 2(12), e1317.
60. Hsiao, E. C., Millard, S. M., Louie, A., Huang, Y., Conklin, B. R., and Nissenson, R. A. (2010). Ligand-mediated activation of an engineered gs g protein-coupled receptor in osteoblasts increases trabecular bone formation. Mol Endocrinol 24(3), 621-631.
61. Hsiao, E. C., Nguyen, T. D., Ng, J. K., Scott, M. J., Chang, W. C., Zahed, H., and Conklin, B. R. (2011). Constitutive Gₛ activation using a single-construct tetracycline-inducible expression system in embryonic stem cells and mice. Stem Cell Res Ther 2(2), 11.
62. Cain, C. J., Valencia, J. T., Ho, S., Jordan, K., Mattingly, A., Morales, B. M., and Hsiao, E. C. (2016). Increased Gₛ Signaling in Osteoblasts Reduces Bone Marrow and Whole-Body Adiposity in Male Mice. Endocrinology 157(4), 1481-1494.
63. Bershteyn, M., Hayashi, Y., Desachy, G., Hsiao, E. C., Sami, S., Tsang, K. M., Weiss, L. A., Kriegstein, A. R., Yamanaka, S., and Wynshaw-Boris, A. (2014). Cell-autonomous correction of ring chromosomes in human induced pluripotent stem cells. Nature 507(7490), 99-103.
64. Niethamer, T. K., Larson, A. R., O'Neill, A. K., Bershteyn, M., Hsiao, E. C., Klein, O. D., Pomerantz, J. H., and Bush, J. O. (2017). EPHRIN-B1 Mosaicism Drives Cell Segregation in Craniofrontonasal Syndrome hiPSC-Derived Neuroepithelial Cells. Stem Cell Reports 8(3), 529-537.
65. Barruet, E., and Hsiao, E. C. (2016). Using Human Induced Pluripotent Stem Cells to Model Skeletal Diseases. Methods Mol Biol 1353, 101-118.
66. Hayashi, Y., Hsiao, E. C., Sami, S., Lancero, M., Schlieve, C. R., Nguyen, T., Yano, K., Nagahashi, A., Ikeya, M., Matsumoto, Y., Nishimura, K., Fukuda, A., Hisatake, K., Tomoda, K., Asaka, I., Toguchida, J., Conklin, B. R., and Yamanaka, S. (2016). BMP-SMAD-ID promotes reprogramming to pluripotency by inhibiting p16/INK4A-dependent senescence. Proc Natl Acad Sci USA 113(46), 13057-13062.
67. Matsumoto, Y., Hayashi, Y., Schlieve, C. R., Ikeya, M., Kim, H., Nguyen, T. D., Sami, S., Baba, S., Barruet, E., Nasu, A., Asaka, I., Otsuka, T., Yamanaka, S., Conklin, B. R., Toguchida, J., and Hsiao, E. C. (2013). Induced pluripotent stem cells from patients with human fibrodysplasia ossificans progressiva show increased mineralization and cartilage formation. Orphanet J Rare Dis 8, 190.
68. Barruet, E., Morales, B. M., Lwin, W., White, M. P., Theodoris, C. V., Kim, H., Urrutia, A., Wong, S. A., Srivastava, D., and Hsiao, E. C. (2016). The ACVR1 R206H mutation found in fibrodysplasia ossificans progressiva increases human induced pluripotent stem cell-derived endothelial cell formation and collagen production through BMP-mediated SMAD1/5/8 signaling. Stem Cell Res Ther 7(1), 115.
69. Yu, X., Ton, A. N., Niu, Z., Morales, B. M., Chen, J., et al. (2022). An ACVR1 activating mutation causes neuropathic pain and sensory neuron hyperexcitability in humans. Pain.
70. Cong, L., Ran, F. A., Cox, D., Lin, S., Barretto, R., Habib, N., Hsu, P. D., Wu, X., Jiang, W., Marraffini, L. A., and Zhang, F. (2013). Multiplex genome engineering using CRISPR/Cas systems. Science 339(6121), 819-823.
71. Tobar-Rubin, R., Sultan, D., Janevska, D., Turcic, K., Carroll, J., Ooms, L., and Pals-Rylaarsdam, R. (2013). Intragenic suppression of a constitutively active allele of Gsalpha associated with McCune-Albright syndrome. Journal of molecular endocrinology 50(2), 193-201.
72. Kuleshov, M. V., Jones, M. R., Rouillard, A. D., Fernandez, N. F., Duan, Q., Wang, Z., Koplev, S., Jenkins, S. L., Jagodnik, K. M., Lachmann, A., McDermott, M. G., Monteiro, C. D., Gundersen, G. W., and Ma'ayan, A. (2016). Enrichr: a comprehensive gene set enrichment analysis web server 2016 update. Nucleic acids research 44(W1), W90-97.
73. Wallach, I., Dzamba, M., and Heifets, A. (2015). AtomNet: A Deep Convolutional Neural Network for Bioactivity Prediction in Structure-based Drug Discovery. Arxivorg(arXiv:1510.02855).

### Sequences

Human GNAS isoform GNASL-Wild-type
(SEQ ID NO: 1)
MGCLGNSKTEDQRNEEKAQREANKKIEKQLQKDKQVYRATHRLLLLGAGESGKSTIVKQMRILHV
NGFNGEGGEEDPQAARSNSDGEKATKVQDIKNNLKEAIETIVAAMSNLVPPVELANPENQFRVDYI
LSVMNVPDFDFPPEFYEHAKALWEDEGVRACYERSNEYQLIDCAQYFLDKIDVIKQADYVPSDQD
LLRCRVLTSGIFETKFQVDKVNFHMFDVGGQRDERRKWIQCFNDVTAIIFVVASSSYNMVIREDNQ
TNRLQEALNLFKSIWNNRWLRTISVILFLNKQDLLAEKVLAGKSKIEDYFPEFARYTTPEDATPEPG
EDPRVTRAKYFIRDEFLRISTASGDGRHYCYPHFTCAVDTENIRRVFNDCRDIIQRMHLRQYELL
NM_000516.7 (GNAS1 human)-Wild-type
(SEQ ID NO: 2)
AGCAGCTCCCGCAGCTCCTGCTCTGGTCCGCCTCGGCCCGGCGGCGGCCATCAGCCCCCTC
GGCCTCGGCTCGAGGGGGGGGGAGCTGCGCGCGCCCCTCGGTCCGACCGACACCCTCCCC
TTCCCGCCCGTCCGCGCGCCCCGCGGCCCGCGGCCCGCAGTCCGCCCCGCGCGCTCCTTG
CCGAGGAGCCGAGCCCGCGCCCGGCCCGCCCGCCCGGCGCTGCCCCGGCCCTCCCGGCC
CGCGTGAGGCCGCCCGCGCCCGCCGCCGCCGCAGCCCGGCCGCGCCCCGCCGCCGCCGC
CGCCGCCATGGGCTGCCTCGGGAACAGTAAGACCGAGGACCAGCGCAACGAGGAGAAGGC
GCAGCGTGAGGCCAACAAAAAGATCGAGAAGCAGCTGCAGAAGGACAAGCAGGTCTACCGG
GCCACGCACCGCCTGCTGCTGCTGGGTGCTGGAGAATCTGGTAAAAGCACCATTGTGAAGCA
GATGAGGATCCTGCATGTTAATGGGTTTAATGGAGAGGGCGGCGAAGAGGACCCGCAGGCT
GCAAGGAGCAACAGCGATGGTGAGAAGGCAACCAAAGTGCAGGACATCAAAAACAACCTGAA
AGAGGCGATTGAAACCATTGTGGCCGCCATGAGCAACCTGGTGCCCCCCGTGGAGCTGGCC
AACCCCGAGAACCAGTTCAGAGTGGACTACATCCTGAGTGTGATGAACGTGCCTGACTTTGA
CTTCCCTCCCGAATTCTATGAGCATGCCAAGGCTCTGTGGGAGGATGAAGGAGTGCGTGCCT
GCTACGAACGCTCCAACGAGTACCAGCTGATTGACTGTGCCCAGTACTTCCTGGACAAGATC
GACGTGATCAAGCAGGCTGACTATGTGCCGAGCGATCAGGACCTGCTTCGCTGCCGTGTCCT
GACTTCTGGAATCTTTGAGACCAAGTTCCAGGTGGACAAAGTCAACTTCCACATGTTTGACGT
GGGTGGCCAGCGCGATGAACGCCGCAAGTGGATCCAGTGCTTCAACGATGTGACTGCCATC
ATCTTCGTGGTGGCCAGCAGCAGCTACAACATGGTCATCCGGGAGGACAACCAGACCAACCG
CCTGCAGGAGGCTCTGAACCTCTTCAAGAGCATCTGGAACAACAGATGGCTGCGCACCATCT
CTGTGATCCTGTTCCTCAACAAGCAAGATCTGCTCGCTGAGAAAGTCCTTGCTGGGAAATCGA
AGATTGAGGACTACTTTCCAGAATTTGCTCGCTACACTACTCCTGAGGATGCTACTCCCGAGC
CCGGAGAGGACCCACGCGTGACCCGGGCCAAGTACTTCATTCGAGATGAGTTTCTGAGGATC
AGCACTGCCAGTGGAGATGGGCGTCACTACTGCTACCCTCATTTCACCTGCGCTGTGGACAC
TGAGAACATCCGCCGTGTGTTCAACGACTGCCGTGACATCATTCAGCGCATGCACCTTCGTCA
GTACGAGCTGCTCTAAGAAGGGAACCCCCAAATTTAATTAAAGCCTTAAGCACAATTAATTAAA
AGTGAAACGTAATTGTACAAGCAGTTAATCACCCACCATAGGGCATGATTAACAAAGCAACCT
TTCCCTTCCCCCGAGTGATTTTGCGAAACCCCCTTTTCCCTTCAGCTTGCTTAGATGTTCCAAA
TTTAGAAAGCTTAAGGCGGCCTACAGAAAAAGGAAAAAAGGCCACAAAAGTTCCCTCTCACTT
TCAGTAAAAATAAATAAAACAGCAGCAGCAAACAAATAAAATGAAATAAAAGAAACAAATGAAA
TAAATATTGTGTTGTGCAGCATTAAAAAAAATCAAAATAAAAATTAAATGTGAGCAAAGAA
NM_00516.7 (GNAS1 human)-p.R201H (c.602G > A)
(SEQ ID NO: 3)
AGCAGCTCCCGCAGCTCCTGCTCTGGTCCGCCTCGGCCCGGCGGCGGCCATCAGCCCCCTC
GGCCTCGGCTCGAGGGGGGGGGAGCTGCGCGCGCCCCTCGGTCCGACCGACACCCTCCCC
TTCCCGCCCGTCCGCGCGCCCCGCGGCCCGCGGCCCGCAGTCCGCCCCGCGCGCTCCTTG
CCGAGGAGCCGAGCCCGCGCCCGGCCCGCCCGCCCGGCGCTGCCCCGGCCCTCCCGGCC
CGCGTGAGGCCGCCCGCGCCCGCCGCCGCCGCAGCCCGGCCGCGCCCCGCCGCCGCCGC
CGCCGCCATGGGCTGCCTCGGGAACAGTAAGACCGAGGACCAGCGCAACGAGGAGAAGGC
GCAGCGTGAGGCCAACAAAAAGATCGAGAAGCAGCTGCAGAAGGACAAGCAGGTCTACCGG
GCCACGCACCGCCTGCTGCTGCTGGGTGCTGGAGAATCTGGTAAAAGCACCATTGTGAAGCA
GATGAGGATCCTGCATGTTAATGGGTTTAATGGAGAGGGCGGCGAAGAGGACCCGCAGGCT
GCAAGGAGCAACAGCGATGGTGAGAAGGCAACCAAAGTGCAGGACATCAAAAACAACCTGAA
AGAGGCGATTGAAACCATTGTGGCCGCCATGAGCAACCTGGTGCCCCCCGTGGAGCTGGCC
AACCCCGAGAACCAGTTCAGAGTGGACTACATCCTGAGTGTGATGAACGTGCCTGACTTTGA
CTTCCCTCCCGAATTCTATGAGCATGCCAAGGCTCTGTGGGAGGATGAAGGAGTGCGTGCCT
GCTACGAACGCTCCAACGAGTACCAGCTGATTGACTGTGCCCAGTACTTCCTGGACAAGATC
GACGTGATCAAGCAGGCTGACTATGTGCCGAGCGATCAGGACCTGCTTCGCTGCCATGTCCT
GACTTCTGGAATCTTTGAGACCAAGTTCCAGGTGGACAAAGTCAACTTCCACATGTTTGACGT
GGGTGGCCAGCGCGATGAACGCCGCAAGTGGATCCAGTGCTTCAACGATGTGACTGCCATC
ATCTTCGTGGTGGCCAGCAGCAGCTACAACATGGTCATCCGGGAGGACAACCAGACCAACCG
CCTGCAGGAGGCTCTGAACCTCTTCAAGAGCATCTGGAACAACAGATGGCTGCGCACCATCT
CTGTGATCCTGTTCCTCAACAAGCAAGATCTGCTCGCTGAGAAAGTCCTTGCTGGGAAATCGA
AGATTGAGGACTACTTTCCAGAATTTGCTCGCTACACTACTCCTGAGGATGCTACTCCCGAGC
CCGGAGAGGACCCACGCGTGACCCGGGCCAAGTACTTCATTCGAGATGAGTTTCTGAGGATC
AGCACTGCCAGTGGAGATGGGCGTCACTACTGCTACCCTCATTTCACCTGCGCTGTGGACAC
TGAGAACATCCGCCGTGTGTTCAACGACTGCCGTGACATCATTCAGCGCATGCACCTTCGTCA
GTACGAGCTGCTCTAAGAAGGGAACCCCCAAATTTAATTAAAGCCTTAAGCACAATTAATTAAA
AGTGAAACGTAATTGTACAAGCAGTTAATCACCCACCATAGGGCATGATTAACAAAGCAACCT
TTCCCTTCCCCCGAGTGATTTTGCGAAACCCCCTTTTCCCTTCAGCTTGCTTAGATGTTCCAAA
TTTAGAAAGCTTAAGGCGGCCTACAGAAAAAGGAAAAAAGGCCACAAAAGTTCCCTCTCACTT
TCAGTAAAAATAAATAAAACAGCAGCAGCAAACAAATAAAATGAAATAAAAGAAACAAATGAAA
TAAATATTGTGTTGTGCAGCATTAAAAAAAATCAAAATAAAAATTAAATGTGAGCAAAGAA
NM_00516.7 (GNAS1 human)-p.R201H (c.602G > A) + silent mutation (c.600 C > T)
(SEQ ID NO: 4)
AGCAGCTCCCGCAGCTCCTGCTCTGGTCCGCCTCGGCCCGGCGGCGGCCATCAGCCCCCTC
GGCCTCGGCTCGAGGGGGGGGGAGCTGCGCGCGCCCCTCGGTCCGACCGACACCCTCCCC
TTCCCGCCCGTCCGCGCGCCCCGCGGCCCGCGGCCCGCAGTCCGCCCCGCGCGCTCCTTG
CCGAGGAGCCGAGCCCGCGCCCGGCCCGCCCGCCCGGCGCTGCCCCGGCCCTCCCGGCC
CGCGTGAGGCCGCCCGCGCCCGCCGCCGCCGCAGCCCGGCCGCGCCCCGCCGCCGCCGC
CGCCGCCATGGGCTGCCTCGGGAACAGTAAGACCGAGGACCAGCGCAACGAGGAGAAGGC
GCAGCGTGAGGCCAACAAAAAGATCGAGAAGCAGCTGCAGAAGGACAAGCAGGTCTACCGG
GCCACGCACCGCCTGCTGCTGCTGGGTGCTGGAGAATCTGGTAAAAGCACCATTGTGAAGCA
GATGAGGATCCTGCATGTTAATGGGTTTAATGGAGAGGGCGGCGAAGAGGACCCGCAGGCT
GCAAGGAGCAACAGCGATGGTGAGAAGGCAACCAAAGTGCAGGACATCAAAAACAACCTGAA
AGAGGCGATTGAAACCATTGTGGCCGCCATGAGCAACCTGGTGCCCCCCGTGGAGCTGGCC
AACCCCGAGAACCAGTTCAGAGTGGACTACATCCTGAGTGTGATGAACGTGCCTGACTTTGA
CTTCCCTCCCGAATTCTATGAGCATGCCAAGGCTCTGTGGGAGGATGAAGGAGTGCGTGCCT
GCTACGAACGCTCCAACGAGTACCAGCTGATTGACTGTGCCCAGTACTTCCTGGACAAGATC
GACGTGATCAAGCAGGCTGACTATGTGCCGAGCGATCAGGACCTGCTTCGCTGTCATGTCCT
GACTTCTGGAATCTTTGAGACCAAGTTCCAGGTGGACAAAGTCAACTTCCACATGTTTGACGT
GGGTGGCCAGCGCGATGAACGCCGCAAGTGGATCCAGTGCTTCAACGATGTGACTGCCATC
ATCTTCGTGGTGGCCAGCAGCAGCTACAACATGGTCATCCGGGAGGACAACCAGACCAACCG
CCTGCAGGAGGCTCTGAACCTCTTCAAGAGCATCTGGAACAACAGATGGCTGCGCACCATCT
CTGTGATCCTGTTCCTCAACAAGCAAGATCTGCTCGCTGAGAAAGTCCTTGCTGGGAAATCGA
AGATTGAGGACTACTTTCCAGAATTTGCTCGCTACACTACTCCTGAGGATGCTACTCCCGAGC
CCGGAGAGGACCCACGCGTGACCCGGGCCAAGTACTTCATTCGAGATGAGTTTCTGAGGATC
AGCACTGCCAGTGGAGATGGGCGTCACTACTGCTACCCTCATTTCACCTGCGCTGTGGACAC
TGAGAACATCCGCCGTGTGTTCAACGACTGCCGTGACATCATTCAGCGCATGCACCTTCGTCA
GTACGAGCTGCTCTAAGAAGGGAACCCCCAAATTTAATTAAAGCCTTAAGCACAATTAATTAAA
AGTGAAACGTAATTGTACAAGCAGTTAATCACCCACCATAGGGCATGATTAACAAAGCAACCT
TTCCCTTCCCCCGAGTGATTTTGCGAAACCCCCTTTTCCCTTCAGCTTGCTTAGATGTTCCAAA
TTTAGAAAGCTTAAGGCGGCCTACAGAAAAAGGAAAAAAGGCCACAAAAGTTCCCTCTCACTT
TCAGTAAAAATAAATAAAACAGCAGCAGCAAACAAATAAAATGAAATAAAAGAAACAAATGAAA
TAAATATTGTGTTGTGCAGCATTAAAAAAAATCAAAATAAAAATTAAATGTGAGCAAAGAA
NM_000516.7 (GNAS1 human)-p.R201C (c.601C > T) + silent mutation (c.600 C > T)
(SEQ ID NO: 5)
AGCAGCTCCCGCAGCTCCTGCTCTGGTCCGCCTCGGCCCGGCGGCGGCCATCAGCCCCCTC
GGCCTCGGCTCGAGGGGGGGGGAGCTGCGCGCGCCCCTCGGTCCGACCGACACCCTCCCC
TTCCCGCCCGTCCGCGCGCCCCGCGGCCCGCGGCCCGCAGTCCGCCCCGCGCGCTCCTTG
CCGAGGAGCCGAGCCCGCGCCCGGCCCGCCCGCCCGGCGCTGCCCCGGCCCTCCCGGCC
CGCGTGAGGCCGCCCGCGCCCGCCGCCGCCGCAGCCCGGCCGCGCCCCGCCGCCGCCGC
CGCCGCCATGGGCTGCCTCGGGAACAGTAAGACCGAGGACCAGCGCAACGAGGAGAAGGC
GCAGCGTGAGGCCAACAAAAAGATCGAGAAGCAGCTGCAGAAGGACAAGCAGGTCTACCGG
GCCACGCACCGCCTGCTGCTGCTGGGTGCTGGAGAATCTGGTAAAAGCACCATTGTGAAGCA
GATGAGGATCCTGCATGTTAATGGGTTTAATGGAGAGGGCGGCGAAGAGGACCCGCAGGCT
GCAAGGAGCAACAGCGATGGTGAGAAGGCAACCAAAGTGCAGGACATCAAAAACAACCTGAA
AGAGGCGATTGAAACCATTGTGGCCGCCATGAGCAACCTGGTGCCCCCCGTGGAGCTGGCC
AACCCCGAGAACCAGTTCAGAGTGGACTACATCCTGAGTGTGATGAACGTGCCTGACTTTGA
CTTCCCTCCCGAATTCTATGAGCATGCCAAGGCTCTGTGGGAGGATGAAGGAGTGCGTGCCT
GCTACGAACGCTCCAACGAGTACCAGCTGATTGACTGTGCCCAGTACTTCCTGGACAAGATC
GACGTGATCAAGCAGGCTGACTATGTGCCGAGCGATCAGGACCTGCTTCGCTGTTGTGTCCT
GACTTCTGGAATCTTTGAGACCAAGTTCCAGGTGGACAAAGTCAACTTCCACATGTTTGACGT
GGGTGGCCAGCGCGATGAACGCCGCAAGTGGATCCAGTGCTTCAACGATGTGACTGCCATC
ATCTTCGTGGTGGCCAGCAGCAGCTACAACATGGTCATCCGGGAGGACAACCAGACCAACCG
CCTGCAGGAGGCTCTGAACCTCTTCAAGAGCATCTGGAACAACAGATGGCTGCGCACCATCT
CTGTGATCCTGTTCCTCAACAAGCAAGATCTGCTCGCTGAGAAAGTCCTTGCTGGGAAATCGA
AGATTGAGGACTACTTTCCAGAATTTGCTCGCTACACTACTCCTGAGGATGCTACTCCCGAGC
CCGGAGAGGACCCACGCGTGACCCGGGCCAAGTACTTCATTCGAGATGAGTTTCTGAGGATC
AGCACTGCCAGTGGAGATGGGCGTCACTACTGCTACCCTCATTTCACCTGCGCTGTGGACAC
TGAGAACATCCGCCGTGTGTTCAACGACTGCCGTGACATCATTCAGCGCATGCACCTTCGTCA
GTACGAGCTGCTCTAAGAAGGGAACCCCCAAATTTAATTAAAGCCTTAAGCACAATTAATTAAA
AGTGAAACGTAATTGTACAAGCAGTTAATCACCCACCATAGGGCATGATTAACAAAGCAACCT
TTCCCTTCCCCCGAGTGATTTTGCGAAACCCCCTTTTCCCTTCAGCTTGCTTAGATGTTCCAAA
TTTAGAAAGCTTAAGGCGGCCTACAGAAAAAGGAAAAAAGGCCACAAAAGTTCCCTCTCACTT
TCAGTAAAAATAAATAAAACAGCAGCAGCAAACAAATAAAATGAAATAAAAGAAACAAATGAAA
TAAATATTGTGTTGTGCAGCATTAAAAAAAATCAAAATAAAAATTAAATGTGAGCAAAGAA
NM_000516.7 (GNAS1 human)-p.R201C (c.601C > T)
(SEQ ID NO: 6)
AGCAGCTCCCGCAGCTCCTGCTCTGGTCCGCCTCGGCCCGGCGGCGGCCATCAGCCCCCTC
GGCCTCGGCTCGAGGGGGGGGGAGCTGCGCGCGCCCCTCGGTCCGACCGACACCCTCCCC
TTCCCGCCCGTCCGCGCGCCCCGCGGCCCGCGGCCCGCAGTCCGCCCCGCGCGCTCCTTG
CCGAGGAGCCGAGCCCGCGCCCGGCCCGCCCGCCCGGCGCTGCCCCGGCCCTCCCGGCC
CGCGTGAGGCCGCCCGCGCCCGCCGCCGCCGCAGCCCGGCCGCGCCCCGCCGCCGCCGC
CGCCGCCATGGGCTGCCTCGGGAACAGTAAGACCGAGGACCAGCGCAACGAGGAGAAGGC
GCAGCGTGAGGCCAACAAAAAGATCGAGAAGCAGCTGCAGAAGGACAAGCAGGTCTACCGG
GCCACGCACCGCCTGCTGCTGCTGGGTGCTGGAGAATCTGGTAAAAGCACCATTGTGAAGCA
GATGAGGATCCTGCATGTTAATGGGTTTAATGGAGAGGGGGCGAAGAGGACCCGCAGGCT
GCAAGGAGCAACAGCGATGGTGAGAAGGCAACCAAAGTGCAGGACATCAAAAACAACCTGAA
AGAGGCGATTGAAACCATTGTGGCCGCCATGAGCAACCTGGTGCCCCCCGTGGAGCTGGCC
AACCCCGAGAACCAGTTCAGAGTGGACTACATCCTGAGTGTGATGAACGTGCCTGACTTTGA
CTTCCCTCCCGAATTCTATGAGCATGCCAAGGCTCTGTGGGAGGATGAAGGAGTGCGTGCCT
GCTACGAACGCTCCAACGAGTACCAGCTGATTGACTGTGCCCAGTACTTCCTGGACAAGATC
GACGTGATCAAGCAGGCTGACTATGTGCCGAGCGATCAGGACCTGCTTCGCTGCTGTGTCCT
GACTTCTGGAATCTTTGAGACCAAGTTCCAGGTGGACAAAGTCAACTTCCACATGTTTGACGT
GGGTGGCCAGCGCGATGAACGCCGCAAGTGGATCCAGTGCTTCAACGATGTGACTGCCATC
ATCTTCGTGGTGGCCAGCAGCAGCTACAACATGGTCATCCGGGAGGACAACCAGACCAACCG
CCTGCAGGAGGCTCTGAACCTCTTCAAGAGCATCTGGAACAACAGATGGCTGCGCACCATCT
CTGTGATCCTGTTCCTCAACAAGCAAGATCTGCTCGCTGAGAAAGTCCTTGCTGGGAAATCGA
AGATTGAGGACTACTTTCCAGAATTTGCTCGCTACACTACTCCTGAGGATGCTACTCCCGAGC
CCGGAGAGGACCCACGCGTGACCCGGGCCAAGTACTTCATTCGAGATGAGTTTCTGAGGATC
AGCACTGCCAGTGGAGATGGGCGTCACTACTGCTACCCTCATTTCACCTGCGCTGTGGACAC
TGAGAACATCCGCCGTGTGTTCAACGACTGCCGTGACATCATTCAGCGCATGCACCTTCGTCA
GTACGAGCTGCTCTAAGAAGGGAACCCCCAAATTTAATTAAAGCCTTAAGCACAATTAATTAAA
AGTGAAACGTAATTGTACAAGCAGTTAATCACCCACCATAGGGCATGATTAACAAAGCAACCT
TTCCCTTCCCCCGAGTGATTTTGCGAAACCCCCTTTTCCCTTCAGCTTGCTTAGATGTTCCAAA
TTTAGAAAGCTTAAGGCGGCCTACAGAAAAAGGAAAAAAGGCCACAAAAGTTCCCTCTCACTT
TCAGTAAAAATAAATAAAACAGCAGCAGCAAACAAATAAAATGAAATAAAAGAAACAAATGAAA
TAAATATTGTGTTGTGCAGCATTAAAAAAAATCAAAATAAAAATTAAATGTGAGCAAAGAA

What is claimed is:

1. A method of screening for an agent that inhibits activity of a hyperactive stimulatory guanine nucleotide binding protein alpha subunit (Gsα) for use in treating a guanine nucleotide-binding protein alpha, stimulatory (GNAS)-associated disorder, the method comprising:
providing an induced pluripotent stem cell (iPSC) comprising a genetically modified GNAS gene comprising an activating mutation, wherein the genetically modified GNAS gene comprises a coding sequence encoding a hyperactive Gsα;
contacting the iPSC with a candidate agent; and
measuring inhibition of the hyperactive Gsα by the candidate agent.

2. The method of claim 1, further comprising:
contacting an iPSC comprising an unmodified GNAS gene encoding a wild-type Gsα with the candidate agent;
measuring inhibition of activity of the wild-type Gsα by the candidate agent;
comparing levels of inhibition of the wild-type Gsα and the hyperactive Gsα, wherein increased inhibition of the hyperactive Gsα compared to the wild-type Gsα indicates that the candidate agent selectively inhibits the hyperactive Gsα.

3. The method of claim 1 or 2, wherein the GNAS-associated disorder is fibrous dysplasia, McCune-Albright syndrome, or cancer.

4. The method of claim 3, wherein the cancer is breast cancer, pancreatic cancer, or adrenal cancer.

5. The method of any one of claims 1-4, wherein the activating mutation is linked to fibrous dysplasia, McCune-Albright syndrome, or cancer.

6. The method of claim 5, wherein the hyperactive Gsα comprises a substitution of histidine or cysteine at amino acid position 201, wherein numbering of amino acid positions is relative to the reference amino acid sequence of SEQ ID NO:1.

7. The method of claim 5, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

8. The method of claim 7, wherein the genetically modified GNAS gene further comprises a substitution of thymine at nucleotide position 600, wherein numbering of nucleotide positions is relative to the reference nucleotide sequence of SEQ ID NO:2.

9. The method of any one of claims 1-8, wherein a CRISPR system is used to genetically modify the genome of the iPSC to introduce the activating mutation into the GNAS gene, wherein a hyperactive Gsα is expressed in the iPSC.

10. The method of claim 9, wherein the CRISPR system comprises a guide RNA (gRNA) capable of hybridizing to a target site in the GNAS gene.

11. The method of any one of claims 1-10, wherein said measuring inhibition of the Gsα comprises measuring inhibition of activation of adenylate cyclase activity by the hyperactive Gsα.

12. The method of claim 11, wherein said measuring inhibition of activation of adenylate cyclase activity comprises measuring levels of intracellular cyclic adenosine monophosphate (cAMP) in presence and absence of the candidate agent, wherein reduced levels of cAMP in the presence of the candidate agent compared to in the absence of the candidate agent indicate that the candidate agent inhibits activation of adenylate cyclase activity by the hyperactive Gsα.

13. The method of any one of claims 1-12, wherein said measuring inhibition of the Gsα comprises measuring inhibition of GTPase activity of the Gsα.

14. The method of any one of claims 1-13, further comprising measuring binding of the candidate agent to the hyperactive Gsα.

15. The method of any one of claims 1-14, further comprising measuring binding of the candidate agent to a G protein-coupled receptor (GPCR) that activates the hyperactive Gsα.

16. The method of any one of claims 1-15, wherein the candidate agent does not fully inhibit the wild-type Gsα.

17. The method of any one of claims 1-16, wherein the agent is a small molecule, a drug, a peptide, a protein, an aptamer, a peptoid, an antibody, or an antibody mimetic.

18. The method of any one of claims 1-17, wherein the iPSC is heterozygous for the genetically modified GNAS gene comprising the activating mutation.

19. The method of any one of claims 1-18, further comprising:
contacting the iPSC with a β-adrenergic receptor agonist; and
measuring inhibition of the hyperactive Gsα by the candidate agent in presence of the β-adrenergic receptor agonist.

20. The method of claim 19, wherein the β-adrenergic receptor agonist is isoproterenol.

21. The method of any one of claims 1-20, further comprising:
contacting the iPSC with a phosphodiesterase inhibitor; and
measuring inhibition of the hyperactive Gsα by the candidate agent in presence of the phosphodiesterase inhibitor.

22. The method of any one of claims 1-21, wherein the genetically modified GNAS gene comprises a nucleotide sequence selected from the group consisting of SEQ ID NOS:3-8.

23. The method of any one of claims 1-21, wherein the genetically modified GNAS gene comprises a nucleotide sequence having at least 90% identity to a sequence selected from the group consisting of SEQ ID NOS:3-8, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

24. A composition comprising:
an induced pluripotent stem cell (iPSC) comprising a genetically modified GNAS gene comprising an activating mutation, wherein the genetically modified GNAS gene comprises a coding sequence encoding a hyperactive stimulatory guanine nucleotide binding protein alpha subunit (Gsα).

25. The composition of claim 24, wherein the activating mutation is linked to fibrous dysplasia, McCune-Albright syndrome, or cancer.

26. The composition of claim 25, wherein the hyperactive Gsα comprises a substitution of histidine or cysteine at amino acid position 201, wherein numbering of amino acid positions is relative to the reference amino acid sequence of SEQ ID NO:1.

27. The composition of claim 25, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.

28. The composition of claim 27, wherein the genetically modified GNAS gene further comprises a substitution of thymine at nucleotide position 600, wherein numbering of nucleotide positions is relative to the reference nucleotide sequence of SEQ ID NO:2.

29. The composition of any one of claims 24-28, further comprising a β-adrenergic receptor agonist.

30. The composition of claim 29, wherein the β-adrenergic receptor agonist is isoproterenol.

31. The composition of any one of claims 24-30, further comprising a phosphodiesterase inhibitor.

32. The composition of any one of claims 24-31, wherein the genetically modified GNAS gene comprises a nucleotide sequence selected from the group consisting of SEQ ID NOS:3-8.

33. The composition of any one of claims 24-31, wherein the genetically modified GNAS gene comprises a nucleotide sequence having at least 90% identity to a sequence selected from the group consisting of SEQ ID NOS:3-8, wherein the genetically modified GNAS gene comprises a substitution of thymine at nucleotide position 601 or adenine at nucleotide position 602, wherein numbering of nucleotide positions is relative to the reference nucleotide sequence of SEQ ID NO:2.


SEQUENCE LISTING (8 sequences)

SEQ ID NO: 1 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 2 - DNA, 1854 bp, Homo sapiens
  source 1..1854: mol_type = genomic DNA
agcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaa

SEQ ID NO: 3 - DNA, 1854 bp, synthetic construct
  source 1..1854: mol_type = other DNA
agcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccatgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaa

SEQ ID NO: 4 - DNA, 1854 bp, synthetic construct
  source 1..1854: mol_type = other DNA
agcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgtcatgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaa

SEQ ID NO: 5 - DNA, 1854 bp, synthetic construct
  source 1..1854: mol_type = other DNA
agcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgttgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaa

SEQ ID NO: 6 - DNA, 1854 bp, synthetic construct
  source 1..1854: mol_type = other DNA
agcagctcccgcagctcctgctctggtccgcctcggcccggcggcggccatcagccccctcggcctcggctcgaggggcggggagctgcgcgcgcccctcggtccgaccgacaccctccccttcccgcccgtccgcgcgccccgcggcccgcggcccgcagtccgccccgcgcgctccttgccgaggagccgagcccgcgcccggcccgcccgcccggcgctgccccggccctcccggcccgcgtgaggccgcccgcgcccgccgccgccgcagcccggccgcgccccgccgccgccgccgccgccatgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgctgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaa

SEQ ID NO: 7 - DNA, 140 bp, synthetic construct
  source 1..140: mol_type = other DNA
ctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtggg

SEQ ID NO: 8 - DNA, 140 bp, synthetic construct
  source 1..140: mol_type = other DNA
gacctgttctagctgcactagttcgtccgactgatacacggctcgctagtcctggacgaagcgacggcacaggactgaagaccttagaaactctggttcaaggtccacctgtttcagttgaaggtgtacaaactgcaccc